SEQUENCE LISTING (87 sequences)

SEQ ID NO: 1 - protein, 16 aa, Artificial Sequence
  Description of Artificial Sequence Synthetic peptide
AKTTPKLEEGEFSEAR

SEQ ID NO: 2 - protein, 17 aa, Artificial Sequence
  Description of Artificial Sequence Synthetic peptide
AKTTPKLEEGEFSEARV

SEQ ID NO: 3 - protein, 9 aa, Artificial Sequence
  Description of Artificial Sequence Synthetic peptide
AKTTPKLGG

SEQ ID NO: 4 - protein, 10 aa, Artificial Sequence
  Description of Artificial Sequence Synthetic peptide
SAKTTPKLGG

SEQ ID NO: 5 - protein, 6 aa, Artificial Sequence
  Description of Artificial Sequence Synthetic peptide
SAKTTP

SEQ ID NO: 6 - protein, 6 aa, Artificial Sequence
  Description of Artificial Sequence Synthetic peptide
RADAAP

SEQ ID NO: 7 - protein, 9 aa, Artificial Sequence
  Description of Artificial Sequence Synthetic peptide
RADAAPTVS

SEQ ID NO: 8 - protein, 12 aa, Artificial Sequence
  Description of Artificial Sequence Synthetic peptide
RADAAAAGGPGS

SEQ ID NO: 9 - protein, 27 aa, Artificial Sequence
  Description of Artificial Sequence Synthetic peptide
RADAAAAGGGGSGGGGSGGGGSGGGGS

SEQ ID NO: 10 - protein, 18 aa, Artificial Sequence
  Description of Artificial Sequence Synthetic peptide
SAKTTPKLEEGEFSEARV

SEQ ID NO: 11 - protein, 5 aa, Artificial Sequence
  Description of Artificial Sequence Synthetic peptide
ADAAP

SEQ ID NO: 12 - protein, 12 aa, Artificial Sequence
  Description of Artificial Sequence Synthetic peptide
ADAAPTVSIFPP

SEQ ID NO: 13 - protein, 5 aa, Artificial Sequence
  Description of Artificial Sequence Synthetic peptide
TVAAP

SEQ ID NO: 14 - protein, 12 aa, Artificial Sequence
  Description of Artificial Sequence Synthetic peptide
TVAAPSVFIFPP

SEQ ID NO: 15 - protein, 6 aa, Artificial Sequence
  Description of Artificial Sequence Synthetic peptide
QPKAAP

SEQ ID NO: 16 - protein, 13 aa, Artificial Sequence
  Description of Artificial Sequence Synthetic peptide
QPKAAPSVTLFPP

SEQ ID NO: 17 - protein, 6 aa, Artificial Sequence
  Description of Artificial Sequence Synthetic peptide
AKTTPP

SEQ ID NO: 18 - protein, 13 aa, Artificial Sequence
  Description of Artificial Sequence Synthetic peptide
AKTTPPSVTPLAP

SEQ ID NO: 19 - protein, 6 aa, Artificial Sequence
  Description of Artificial Sequence Synthetic peptide
AKTTAP

SEQ ID NO: 20 - protein, 13 aa, Artificial Sequence
  Description of Artificial Sequence Synthetic peptide
AKTTAPSVYPLAP

SEQ ID NO: 21 - protein, 6 aa, Artificial Sequence
  Description of Artificial Sequence Synthetic peptide
ASTKGP

SEQ ID NO: 22 - protein, 13 aa, Artificial Sequence
  Description of Artificial Sequence Synthetic peptide
ASTKGPSVFPLAP

SEQ ID NO: 23 - protein, 15 aa, Artificial Sequence
  Description of Artificial Sequence Synthetic peptide
GGGGSGGGGSGGGGS

SEQ ID NO: 24 - protein, 15 aa, Artificial Sequence
  Description of Artificial Sequence Synthetic peptide
GENKVEYAPALMALS

SEQ ID NO: 25 - protein, 15 aa, Artificial Sequence
  Description of Artificial Sequence Synthetic peptide
GPAKELTPLKEAKVS

SEQ ID NO: 26 - protein, 15 aa, Artificial Sequence
  Description of Artificial Sequence Synthetic peptide
GHEAAAVMQVQYPAS

SEQ ID NO: 27 - protein, 24 aa, Artificial Sequence
  Description of Artificial Sequence Synthetic peptide
TVAAPSVFIFPPTVAAPSVFIFPP

SEQ ID NO: 28 - protein, 26 aa, Artificial Sequence
  Description of Artificial Sequence Synthetic peptide
ASTKGPSVFPLAPASTKGPSVFPLAP

SEQ ID NO: 29 - protein, 10 aa, Artificial Sequence
  Description of Artificial Sequence Synthetic peptide
GGGGSGGGGS

SEQ ID NO: 30 - protein, 9 aa, Artificial Sequence
  Description of Artificial Sequence Synthetic peptide
GGSGGGGSG

SEQ ID NO: 31 - protein, 5 aa, Artificial Sequence
  Description of Artificial Sequence Synthetic peptide
GGGGS

SEQ ID NO: 32 - protein, 120 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 33 - protein, 113 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 34 - protein, 121 aa, Artificial Sequence
  Description of Artificial Sequence Synthetic polypeptide
QVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSS

SEQ ID NO: 35 - protein, 107 aa, Artificial Sequence
  Description of Artificial Sequence Synthetic polypeptide
NTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKR

SEQ ID NO: 36 - protein, 116 aa, Artificial Sequence
  Description of Artificial Sequence Synthetic polypeptide
EVQLQQSGPEPAKPGASVKMSCKASGYTFTSSVIHWVKQKPGQGLEWIGYINPYNDDTKYNEKFKGKATLTSDKSSSTAYMELSSLTSEDSGVYYCARRLRLFAYWGQGTLVTVSA

SEQ ID NO: 37 - protein, 109 aa, Artificial Sequence
  Description of Artificial Sequence Synthetic polypeptide
EIVLTQSPALMAASPGEKVTITCSVSSSISSSNLHWYQQKSESSPKPWIYGTSNLASGVPVRFSGSGSGTSYSLTISSMEAEDAATYYCQQWSGYPLTFGSGTKLEIKR

SEQ ID NO: 38 - protein, 118 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSS

SEQ ID NO: 39 - protein, 114 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKR

SEQ ID NO: 40 - protein, 244 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSSGGGGSGGGGSEVQLQQSGPEPAKPGASVKMSCKASGYTFTSSVIHWVKQKPGQGLEWIGYINPYNDDTKYNEKFKGKATLTSDKSSSTAYMELSSLTSEDSGVYYCARRLRLFAYWGQGTLVTVSA

SEQ ID NO: 41 - protein, 232 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKRGGSGGGGSGEIVLTQSPALMAASPGEKVTITCSVSSSISSSNLHWYQQKSESSPKPWIYGTSNLASGVPVRFSGSGSGTSYSLTISSMEAEDAATYYCQQWSGYPLTFGSGTKLEIKR

SEQ ID NO: 42 - protein, 240 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSSASTKGPEVQLQQSGPEPAKPGASVKMSCKASGYTFTSSVIHWVKQKPGQGLEWIGYINPYNDDTKYNEKFKGKATLTSDKSSSTAYMELSSLTSEDSGVYYCARRLRLFAYWGQGTLVTVSA

SEQ ID NO: 43 - protein, 228 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKRTVAAPEIVLTQSPALMAASPGEKVTITCSVSSSISSSNLHWYQQKSESSPKPWIYGTSNLASGVPVRFSGSGSGTSYSLTISSMEAEDAATYYCQQWSGYPLTFGSGTKLEIKR

SEQ ID NO: 44 - protein, 247 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSSASTKGPSVFPLAPEVQLQQSGPEPAKPGASVKMSCKASGYTFTSSVIHWVKQKPGQGLEWIGYINPYNDDTKYNEKFKGKATLTSDKSSSTAYMELSSLTSEDSGVYYCARRLRLFAYWGQGTLVTVSA

SEQ ID NO: 45 - protein, 235 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKRTVAAPSVFIFPPEIVLTQSPALMAASPGEKVTITCSVSSSISSSNLHWYQQKSESSPKPWIYGTSNLASGVPVRFSGSGSGTSYSLTISSMEAEDAATYYCQQWSGYPLTFGSGTKLEIKR

SEQ ID NO: 46 - protein, 247 aa, Artificial Sequence; identical to SEQ ID NO: 44
  Description of Artificial Sequence Synthetic polypeptide

SEQ ID NO: 47 - protein, 228 aa, Artificial Sequence; identical to SEQ ID NO: 43
  Description of Artificial Sequence Synthetic polypeptide

SEQ ID NO: 48 - protein, 251 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSSGGGGSGGGGSQVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSS

SEQ ID NO: 49 - protein, 229 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKRGGSGGGGSGNTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKR

SEQ ID NO: 50 - protein, 247 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSSASTKGPQVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSS

SEQ ID NO: 51 - protein, 225 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKRTVAAPNTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKR

SEQ ID NO: 52 - protein, 254 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSSASTKGPSVFPLAPQVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSS

SEQ ID NO: 53 - protein, 232 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKRTVAAPSVFIFPPNTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKR

SEQ ID NO: 54 - protein, 247 aa, Artificial Sequence; identical to SEQ ID NO: 50
  Description of Artificial Sequence Synthetic polypeptide

SEQ ID NO: 55 - protein, 232 aa, Artificial Sequence; identical to SEQ ID NO: 53
  Description of Artificial Sequence Synthetic polypeptide

SEQ ID NO: 56 - protein, 254 aa, Artificial Sequence; identical to SEQ ID NO: 52
  Description of Artificial Sequence Synthetic polypeptide

SEQ ID NO: 57 - protein, 225 aa, Artificial Sequence; identical to SEQ ID NO: 51
  Description of Artificial Sequence Synthetic polypeptide

SEQ ID NO: 58 - protein, 251 aa, Artificial Sequence
  Description of Artificial Sequence Synthetic polypeptide
QVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSSGGGGSGGGGSEVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 59 - protein, 229 aa, Artificial Sequence
  Description of Artificial Sequence Synthetic polypeptide
NTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKRGGSGGGGSGDVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 60 - protein, 247 aa, Artificial Sequence
  Description of Artificial Sequence Synthetic polypeptide
QVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSSASTKGPEVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 61 - protein, 225 aa, Artificial Sequence
  Description of Artificial Sequence Synthetic polypeptide
NTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKRTVAAPDVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 62 - protein, 254 aa, Artificial Sequence
  Description of Artificial Sequence Synthetic polypeptide
QVQLKESGPGLVKPSLTLSLTCTVSGFSLNGYGVIWVRQPPGKGLEWMGVIWGNGNTNYNSTLKSRLSISRDTSKSQVFLKMNNLQTEDTAMYFCARSENYYSSPGYFAYWGQGTLVTVSSASTKGPSVFPLAPEVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 63 - protein, 232 aa, Artificial Sequence
  Description of Artificial Sequence Synthetic polypeptide
NTVMTQSPTSMFISVGDRVTMNCKASQNVGSDVDWYQQKTGQSPKLLISGTSNRYTGVPDRFTGSGSGTDFTLTISNMQAEDLAVYYCLQYNYNPTFGAGTKLELKRTVAAPSVFIFPPDVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 64 - protein, 247 aa, Artificial Sequence; identical to SEQ ID NO: 60
  Description of Artificial Sequence Synthetic polypeptide

SEQ ID NO: 65 - protein, 232 aa, Artificial Sequence; identical to SEQ ID NO: 63
  Description of Artificial Sequence Synthetic polypeptide

SEQ ID NO: 66 - protein, 254 aa, Artificial Sequence; identical to SEQ ID NO: 62
  Description of Artificial Sequence Synthetic polypeptide

SEQ ID NO: 67 - protein, 225 aa, Artificial Sequence; identical to SEQ ID NO: 61
  Description of Artificial Sequence Synthetic polypeptide

SEQ ID NO: 68 - protein, 248 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSSGGGGSGGGGSQVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSS

SEQ ID NO: 69 - protein, 236 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKRGGSGGGGSGDIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKR

SEQ ID NO: 70 - protein, 244 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSSASTKGPQVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSS

SEQ ID NO: 71 - protein, 232 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKRTVAAPDIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKR

SEQ ID NO: 72 - protein, 244 aa, Artificial Sequence; identical to SEQ ID NO: 70
  Description of Artificial Sequence Synthetic polypeptide

SEQ ID NO: 73 - protein, 239 aa, Artificial Sequence
  Description of Artificial Sequence Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKRTVAAPSVFIFPPDIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKR

SEQ ID NO: 74 - protein, 251 aa, Artificial Sequence
  Description of Artificial Sequence Synthetic polypeptide
EVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSSASTKGPSVFPLAPQVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSS

SEQ ID NO: 75 - protein, 232 aa, Artificial Sequence; identical to SEQ ID NO: 71
  Description of Artificial Sequence Synthetic polypeptide

SEQ ID NO: 76 - protein, 248 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSSGGGGSGGGGSEVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 77 - protein, 236 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKRGGSGGGGSGDVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 78 - protein, 244 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSSASTKGPEVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 79 - protein, 232 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKRTVAAPDVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 80 - protein, 244 aa, Artificial Sequence; identical to SEQ ID NO: 78
  Description of Artificial Sequence Synthetic polypeptide

SEQ ID NO: 81 - protein, 239 aa, Artificial Sequence
  Description of Artificial Sequence Synthetic polypeptide
DIVMTQSPSSLAMSVGQKVTMSCKSSQSLLNSSNQKNYLAWYQQKPGQSPELLVYFASTRESGVPDRFIGSGSGTDFTLTISSVQAEDLADYFCQQHYSTPFTFGSETKLEIKRTVAAPSVFIFPPDVVMTQTPLSLPVSLGDQASISCRSSQSLVHSNGNTYLHWYLQKPGQSPKLLIYKVSNRFSGVPDRFSGSGSGTDFTLKISRVEAEDLGAYFCSQSTHVPYTFGGPTRLEIKR

SEQ ID NO: 82 - protein, 251 aa, Artificial Sequence
  Description of Artificial Sequence Synthetic polypeptide
QVQLQQPGAEFVKPGAPVKLSCKASGYPFTTYWVNWMKQRPGRGLEWIGRIDPYDSETLYNQKFKDKATLTVDKSSSTAYIQLSSLTSEDSAVYYCARETYDYPFAYWGQGTLVTVSSASTKGPSVFPLAPEVQLVESGGGLVKPGGSLKLSCVASGFTFSTYAMSWVRQTPTKRLEWVATISRGGVSTYYPDTVKGRFTISRDNAKNTLYLQMSSLRSEDTAMYYCARPPTIVTTWFAYWGQGTLVTVSS

SEQ ID NO: 83 - protein, 232 aa, Artificial Sequence; identical to SEQ ID NO: 79
  Description of Artificial Sequence Synthetic polypeptide

SEQ ID NO: 84 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2015/0125397 A1
(43) Pub. Date: May 7, 2015

(54) DUAL SPECIFIC BINDING PROTEINS DIRECTED AGAINST IMMUNE CELL RECEPTORS AND AUTOANTIGENS

(71) Applicants: ABBVIE INC., NORTH CHICAGO, IL (US); UNIVERSITY OF MACHUSETTS, WORCESTER, MA (US)

(72) Inventors: Chee-Ho Choi, Jefferson, MA (US); Tariq Ghayur, Holliston, MA (US); Ann Marshak-Rothstein, Newton, MA (US); Krishna Moody, Worcester, MA (US)

(21) Appl. No.: 14/507,719

(22) Filed: Oct. 6, 2014

Related U.S. Application Data
(60) Provisional application No. 61/887,412, filed on Oct. 6, 2013.
(60) Provisional application No. 61/987,587, filed on May 2, 2014.

(51) Int. Cl. C07K 16/28; A61K 45/06; G01N 33/564; A61K 49/00; G01N 33/50; A61K 39/395; A61K 47/48

(52) U.S. Cl. CPC C07K 16/2866; C07K 16/28; A61K 39/39533; A61K 39/3955; A61K 45/06; A61K 47/48384; A61K 47/48561; A61K 49/0058; G01N 33/5052; G01N 33/564; C07K 2317/31; C07K 2317/74; C07K 2319/85; C07K 2319/80; G01N 2500/10; G01N 2800/52; G01N 2333/705
USPC 424/9.6; 530/387.3; 530/391.1; 530/391.7; 424/136.1; 424/178.1; 536/23.53; 424/133.1; 435/320.1; 435/69.6; 435/252.33; 435/419; 435/254.11; 435/328; 435/254.21; 435/7.24

(57) ABSTRACT

Engineered multivalent and multispecific binding proteins that bind immune cell receptors and/or autoantigens are provided, along with methods of making and uses in the prevention, diagnosis, prognosis and/or treatment of disease.

### RELATED APPLICATION

This application claims priority from U.S. Provisional Patent Application No. 61/887,412, filed on Oct. 6, 2013 and U.S. Provisional Patent Application No. 61/987,587, filed on May 2, 2014, both of which are incorporated by reference, herein in their entireties.

### FIELD

Multivalent and multispecific binding proteins that bind B cell receptors and autoantigens, methods of making, and their uses, including the diagnosis, prognosis, prevention, and treatment of autoimmune disease, as well as the screening of therapeutics and clinical trial candidates, are provided.

### BACKGROUND OF THE INVENTION

Autoimmune diseases are a common health problem, yet the etiologies of these diseases are still poorly understood. Autoimmune diseases can be classified into two broad, but overlapping, categories: organ-specific and systemic. In organ-specific autoimmune disease, local injury, inflammation, or dysfunction are produced by autoantibody- or cell-mediated reactions against a specific target antigen located in a specialized cell, tissue, or organ. In contrast, systemic autoimmune disease involves tissue injury and inflammation at multiple sites without regard to the autoantigenic insult and is usually initiated by vascular leakage and deposition of circulating autologous immune complexes (ICs). These ICs are formed by autoantibody responses to ubiquitous soluble cellular self antigens of nuclear or, less commonly, cytoplasmic origin. Systemic Lupus Erythematosus (SLE), Rheumatoid Arthritis (RA), Sjogren's Syndrome (SS), Progressive Systemic Sclerosis (PSS), and Mixed Connective Tissue Disease (MCTD) are examples of such debilitating IC-mediated systemic autoimmune diseases.

SLE, for example, is characterized by dysregulation of the immune system resulting in the production of antinuclear antibodies and the generation of circulating immune complexes. These immune complexes build up in tissues and joints, causing their inflammation and degradation. The disease affects most organ systems, if not the entire body, and often involves inflammation and consequent injury to the joints, skin, kidney, brain, body cavity membranes, lung, heart, and gastrointestinal tract. The pathologic hallmark of the disease is recurrent, widespread, and diverse vascular lesions resembling a rash or other changes on the surface of the skin.

The precise cause of SLE is unknown. However, it is generally accepted that the disease is caused, either directly or indirectly, by autoantibody production and the subsequent formation of pathogenic ICs. These autoantibodies, which are produced by dysregulated B lymphocytes, have distinct specificities for nuclear autoantigens, including DNA, nucleosomes and subnucleosomes. Additional autoantigenic specificities include certain RNA/protein complexes, such as the Sm antigen and small nuclear ribonucleoproteins (snRNP).

In the context of autoimmune disease, autoantigens circulate as autoantibody-bound ICs that are recognized by IgG-reactive or rheumatoid factor (RF) expressing B cells. Many autoantigens trigger systemic autoimmune disease by associating with macromolecular complexes that stimulate cytosolic innate immune receptors, such as certain Toll-like receptors (TLRs). In the case of autoreactive B cells, the B cell receptor (BCR) binds to the autoantigen and delivers it to an autoantigen-reactive TLR in the appropriate cellular compartment. For example, autoantigens associated with RNA or DNA, e.g., histones or chromatin, can be recognized by the nucleic acid sensing TLR7 or TLR9, respectively, found in endolysosomal compartments. Detection of the associated nucleic acids by the TLR provides a second signal, such as cytokine or transcription factor production, which then promotes B cell activation, leading to the production of autoantibodies.

The idea that BCR delivery of TLR agonists can promote autoreactive B cell activation initially emerged from in vitro studies (Lau et al. (2005) J. Exp. Med. 202(9):1171-7; Leadbetter et al. (2002) Nature 416:603-607), and has subsequently been supported by numerous in vivo observations. For example, TLR7-deficient mice fail to make autoantibodies reactive with RNA-associated autoantigens and TLR9-deficient autoimmune prone mice fail to make autoantibodies reactive with dsDNA or chromatin (Christensen et al. (2005) J. Exp. Med. 202:321-331; Christensen et al. (2006) Immunity 25:417-428; Lartigue et al. (2006) J. Immunol. 177:1349-1354; Nickerson et al. (2010) J. Immunol. 184:1840-1848; Santiago-Raber et al. (2010) J. Autoimmun 34:339-348; Yu et al. (200) Int. Immunol 18:1211-1219). Moreover, autoimmune prone mice lacking only TLR7 have markedly attenuated disease (Christensen et al. (2006) Immunity 25:417-428), while overexpression of TLR7 results in exacerbated clinical symptoms and accelerated mortality (Deane et al. (2007) Immunity 25:417-428; Pisitkun et al. (2006) Science 312:1669-1672; Subramanian et al. (2006) Proc. Natl. Acad. Sci. USA 103:9970-9975). Paradoxically, autoimmune prone mice that fail to express a functional form of TLR9 invariably develop more severe clinical disease and also have a shortened lifespan (Christensen et al. (2005) J. Exp. Med. 202:321-331; Lartigue et al. (2006) J. Immunol. 177:1349-1354; Nickerson et al. (2010) J. Immunol. 184:1840-1848; Santiago-Raber et al. (2010) J. Autoimmun 34:339-348; Yu et al. (2006) Int. Immunol. 18:1211-1219).

Little is known about the differential outcomes of TLR7 and TLR9 engagement or how TLR9, and not TLR7, can mitigate systemic autoimmunity. One of the limiting factors for determining the roles of TLR7 and TLR9 engagement in autoimmunity is the paucity of agents that allow for the intracellular delivery of ICs and activation of TLRs (e.g., TLR7 and TLR9) in immune cells. Targeted triggering of TLR pathways can provide novel therapies as well as prognostics that can inform the selection of clinical trial candidates that may be predisposed to benefit from treatment. However, initial attempts at reproducing the effect of ligating BCR with TLR9 using F(ab′)2 anti-mouse IgM (anti-IgM) to crosslink BCR and CpG DNA to stimulate TLR-9 failed to recapitulate all of the aspects of stimulation with spontaneous immune complexes (Chaturvedi et al. (2008) Immunity 28(6):799-809).

Accordingly, a need exists for novel compositions and methods that can deliver ICs to immune cells and modulate TLR signaling.

### SUMMARY OF THE INVENTION

There is a need in the art for improved multivalent binding proteins capable of binding immune cell receptors and autoantigens. This disclosure provides bispecific binding proteins that bind to a Toll like receptor (TLR)-signaling (e.g., activating or inhibiting) autoantigen, e.g., an RNA or DNA containing autoantigen, and an immune cell receptor, e.g., the B cell receptor, to form an immune complex that is internalized and transported to TLRs resident in the endosomal compartment. The bispecific binding proteins are useful as a vehicle for the modulation of endosomal TLR signaling, and, hence, modulation of autoimmune disease.

In one aspect, the invention provides a bispecific binding protein that binds to at least two targets, wherein target one comprises a TLR-activating autoantigen and target two comprises an immune cell receptor.

In certain embodiments, the TLR-activating autoantigen comprises a deoxyribonucleic acid (DNA) and a ribonucleic acid (RNA).

In certain embodiments, the immune cell receptor comprises a surface bound immunoglobulin or fragment thereof.

In certain embodiments, the immune cell target comprises a B cell.

In certain embodiments, the immune cell receptor comprises a B cell receptor (BCR).

In certain embodiments, the immune cell receptor comprises an IgM immunoglobulin.

In certain embodiments, the immune cell receptor comprises an IgD, IgE, IgA, or IgG immunoglobulin, an immunoglobulin light chain, an immunoglobulin heavy chain, an allotypic immunoglobulin, or an idiotypic immunoglobulin.

In certain embodiments, the TLR comprises TLR7 or TLR9.

In certain embodiments, the bispecific binding protein can cause cell proliferation and/or cell death.

In certain embodiments, the binding protein comprises a format, e.g., a DVD-Ig™ molecule, a BiTe® molecule, a DART® molecule, a DuoBody™ molecule, a scFv/diabody-IgG molecule, a cross-over multispecific (e.g., bispecific) molecule, a 2-in-1 bispecific molecule, a knob-in-hole multispecific (e.g., bispecific) molecule, a CovXBody molecule, an affibody molecule, a scFV/diabody-CH2/CH3 bispecific molecule, a IgG-non-Ig protein scaffold-based multispecific (e.g., bispecific) molecule, a Fynomer®, and a scFV/diabody linked to normal human protein like human serum albumin-bispecific molecule.

In certain embodiments, the DVD-Ig™ molecule has the binding protein framework disclosed in U.S. Pat. No. 7,612,181 (incorporated herein by reference in its entirety) containing a first and a second polypeptide chain, each comprising first and second variable domain sequences (e.g., those listed in Table 1) that form functional binding domain targets, i.e., binding sites for immune cell receptors and autoantigens.

In certain embodiments, a binding protein is disclosed comprising first and second polypeptide chains, each independently comprising the format VD1-(X1)n-VD2-C-(X2)n, wherein: VD1 is a first variable domain, VD2 is a second variable domain, C is a constant domain, X1 is a linker, X2 is an Fc region, n is 0 or 1, and wherein the VD1 domains on the first and second polypeptide chains form a first functional target binding site and the VD2 domains on the first and second polypeptide chains form a second functional target binding site.

In certain embodiments, X1 is a linker with the proviso that it is not CH1 or CL.

In certain embodiments, the bispecific binding protein comprises two first polypeptide chains and two second polypeptide chains that form four functional target binding sites.

In certain embodiments, the binding protein is capable of binding an immune cell receptor and/or an autoantigen. In certain embodiments, the binding protein is capable of binding an immune cell receptor and/or autoantigen with high affinity.

In certain embodiments, the binding protein comprises a polypeptide chain that binds an immune cell receptor and/or an autoantigen, wherein the polypeptide chain comprises the format VD1-(X1)n-VD2-C-(X2)n, wherein VD1 is a first variable domain, VD2 is a second variable domain, C is a constant domain, X1 represents an amino acid or polypeptide, X2 represents an Fc region, and n is 0 or 1. In certain embodiments, the VD1 and/or VD2 in the binding protein are heavy chain variable domains. In certain embodiments, the VD1 and/or VD2 in the binding protein are light chain variable domains. In certain embodiments, X1 is a linker with the proviso that it is not CH1. In certain embodiments, X1 is a linker with the proviso that it is not CL. In still certain embodiments, C is a heavy chain constant domain.

In certain embodiments, the binding proteins disclosed herein comprise a polypeptide chain that binds an immune cell receptor and/or an autoantigen, wherein the polypeptide chain comprises the format VD1-(X1)n-VD2-C-(X2)n, wherein VD1 is a first heavy chain variable domain, VD2 is a second heavy chain variable domain, C is a heavy chain constant domain, X1 is a linker, and X2 is an Fc region. In certain embodiments, X1 is a linker with the proviso that it is not CH1. In certain embodiments, X1 is a linker with the proviso that it is not CL.

In certain embodiments, the binding protein disclosed herein comprises a polypeptide chain that binds an immune cell receptor and/or an autoantigen, wherein the polypeptide chain comprises the format VD1-(X1)n-VD2-C-(X2)n, wherein VD1 is a first light chain variable domain, VD2 is a second light chain variable domain, C is a light chain constant domain, X1 is a linker, and X2 does not comprise an Fc region. In certain embodiments, X1 is a linker with the proviso that it is not CH1. In certain embodiments, X1 is a linker with the proviso that it is not CL.

In certain embodiments, a binding protein that binds an immune cell receptor and/or an autoantigen comprising two polypeptide chains, wherein the first polypeptide chain comprises the format VD1-(X1)n-VD2-C-(X2)n, wherein VD1 is a first variable domain, VD2 is a second variable domain, C is a constant domain, X1 is a first linker, and X2 is an Fc region; and the second polypeptide chain comprises the format VD1-(X1)n-VD2-C-(X2)n, wherein VD1 is a first variable domain, VD2 is a second variable domain, C is a constant domain, X1 is a second linker, and X2 does not comprise an Fc region is provided. In various embodiments, first variable domain is a heavy chain variable domain or a light chain variable domain. In various embodiments, second variable domain is a heavy chain variable domain or a light chain variable domain.

In certain embodiments, a binding protein that binds an immune cell receptor and/or an autoantigen comprising two polypeptide chains, wherein the first polypeptide chain comprises the format VD1-(X1)n-VD2-C-(X2)n, wherein VD1 is a first heavy chain variable domain, VD2 is a second heavy chain variable domain, C is a heavy chain constant domain, X1 is a first linker, and X2 is an Fc region; and the second polypeptide chain comprises the format VD1-(X1)n-VD2-C-(X2)n, wherein VD1 is a first light chain variable domain, VD2 is a second light chain variable domain, C is a light chain constant domain, X1 is a second linker, and X2 does not comprise an Fc region is provided.

In certain embodiments, the first and second X1 are the same. In certain embodiments, the first and second X1 are different. In certain embodiments the first X1 and/or second X1 is not a CH1 domain and/or the first X1 and/or the second X1 is not a CL domain. In certain embodiments, the first X1 and the second X1 are short (e.g., about 6 amino acid) linkers. In certain embodiments, the first X1 and the second X1 are long (e.g., greater than about 6 amino acid) linkers. In certain embodiments, the first X1 is a short linker and the second X1 is a long linker. In certain embodiments, the first X1 is a long linker and the second X1 is a short linker.

In certain embodiments, the disclosure provides a Dual Variable Domain Immunoglobulin (DVD-Ig) molecule comprising four polypeptide chains, wherein each of the first two polypeptide chains comprises the format VD1-(X1)n-VD2-C-(X2)n, wherein VD1 is a first variable domain, VD2 is a second variable domain, C is a constant domain, X1 is a first linker, and X2 is an Fc region; and each of the second two polypeptide chain comprises the format VD1-(X1)n-VD2-C-(X2)n, wherein VD1 is a first variable domain, VD2 is a second variable domain, C is a constant domain, X1 is a second linker, and X2 does not comprise an Fc region. Such a DVD-Ig binding protein has four antigen binding sites. In certain embodiments, the first and second X1 are the same. In certain embodiments, the first and second X1 are different. In certain embodiments, the first X1 and/or second X1 is not a CH1 domain and/or the first X1 and/or the second X1 is not a CL domain.

In certain embodiments, the disclosure provides a DVD-Ig binding protein comprising four polypeptide chains, wherein each of the first two polypeptide chains comprises the format VD1-(X1)n-VD2-C-(X2)n, wherein VD1 is a first heavy chain variable domain, VD2 is a second heavy chain variable domain, C is a heavy chain constant domain, X1 is a first linker, and X2 is an Fc region; and each of the second two polypeptide chain comprises the format VD1-(X1)n-VD2-C-(X2)n, wherein VD1 is a first light chain variable domain, VD2 is a second light chain variable domain, C is a light chain constant domain, X1 is a second linker, and X2 does not comprise an Fc region. Such a DVD-Ig binding protein has four antigen binding sites. In certain embodiments, the first and second X1 are the same. In certain embodiments, the first and second X1 are different. In certain embodiments, the first X1 and/or second X1 is not a CH1 domain and/or the first X1 and/or the second X1 is not a CL domain.

In certain embodiments, the binding proteins comprise at least two variable domain sequences (e.g., VD1 and VD2) capable of binding an immune cell receptor and/or an autoantigen, in any orientation. In certain embodiments, the disclosure provides a binding protein comprising first and second polypeptide chains, each independently comprising format VD1-(X1)n-VD2-C-(X2)n, wherein VD1 is a first variable domain, VD2 is a second variable domain, C is a constant domain, X1 is a linker with the proviso that it is not CH1, X2 is an Fc region, n is 0 or 1, wherein the VD1 domains on the first and second polypeptide chains form a first functional target binding site and the VD2 domains on the first and second polypeptide chains form a second functional target binding site, and wherein the binding protein is capable of binding an immune cell receptor and/or an autoantigen, wherein (i) the variable domains that form a functional target binding site for mouse IgM comprise a sequence selected from the group consisting of SEQ ID NOs: 34 and 35 and/or the binding protein is capable of binding mouse IgM with an EC50 of about 0.19 nM, or about 0.20 nM, or about 0.12 nM, or about 0.10 nM, or about 0.03, or about 0.04, as measured in an IgM binding ELISA, and/or (ii) the variable domains that form a functional target binding site for DNA comprise a sequence selected from the group consisting of SEQ ID NO: 32 and 33, and/or the binding protein has an antinuclear antibody (ANA) score of about 2, or about 3.5, or about 4, or about 4.5.

In certain embodiments, the disclosure provides a binding protein comprising first and second polypeptide chains, each independently comprising the format VD1-(X1)n-VD2-C-(X2)n, wherein VD1 is a first variable domain; VD2 is a second variable domain; C is a constant domain; X1 is a linker with the proviso that it is not CH1 or CL; X2 is an Fc region; n is 0 or 1, wherein the VD1 domains on the first and second polypeptide chains form a first functional target binding site and the VD2 domains on the first and second polypeptide chains form a second functional target binding site, and wherein (a) the binding protein is capable of binding IgM and DNA, wherein (i) the variable domains that form a functional target binding site for IgM comprise: three CDRs (or CDRS 1-3) from the amino acid sequence of SEQ ID NO: 34 and three CDRs (or CDRS 1-3) from the amino acid sequence of SEQ ID NO: 35; and/or the binding protein is capable of binding mouse IgM with an IC50 of about 0.19 nM, or about 0.20 nM, or about 0.12 nM, or about 0.10 nM, or about 0.03, or about 0.04, as measured in an mouse IgM binding ELISA, and/or (ii) the variable domains that form a functional target binding site for DNA comprise three CDRs (or CDRS 1-3) from the amino acid sequence of SEQ ID NO: 32 and three CDRs (or CDRS 1-3) from the amino acid sequence of SEQ ID NO: 33; and/or the binding protein is capable of binding DNA has an ANA score of about 2, or about 3.5, or about 4, or about 4.5.

In certain embodiments, the disclosure provides a binding protein wherein the first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; n is 0 or 1, and wherein the second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1 or CL; X2 does not comprise an Fc region; n is 0 or 1, wherein the VD1 domains on the first and second polypeptide chains form a first functional target binding site for mouse IgM and the VD2 domains on the first and second polypeptide chains form a second functional target binding site for DNA; or wherein the VD1 domains on the first and second polypeptide chains form a first functional target binding site for DNA and the VD2 domains on the first and second polypeptide chains form a second functional target binding site for mouse IgM.

In certain embodiments, (a) the binding protein is capable of binding mouse IgM and DNA, wherein (i) the variable domains that form a functional target binding site for mouse IgM comprise SEQ ID NO: 34 and SEQ ID NO: 35; and/or (ii) the variable domains that form a functional target binding site for DNA comprise SEQ ID NO: 32 and SEQ ID NO: 33. In certain embodiments, the binding protein comprises two first polypeptide chains and two second polypeptide chains, wherein the binding protein comprises four functional target binding sites. In certain embodiments, the disclosure provides a binding protein capable of binding mouse IgM and DNA, wherein the binding protein comprises any one of: DVD3746 (comprising SEQ ID NO: 40 for the heavy chain and SEQ ID NO:41 for the light chain); DVD3747 (comprising SEQ ID NO: 42 for the heavy chain and SEQ ID NO:43 for the light chain); DVD3749 (comprising SEQ ID NO: 44 for the heavy chain and SEQ ID NO:45 for the light chain); DVD3750 (comprising SEQ ID NO: 46 for the heavy chain and SEQ ID NO:47 for the light chain); DVD3751 (comprising SEQ ID NO: 48 for the heavy chain and SEQ ID NO:49 for the light chain); DVD3752 (comprising SEQ ID NO: 50 for the heavy chain and SEQ ID NO: 51 for the light chain); DVD3753 (comprising SEQ ID NO: 52 for the heavy chain and SEQ ID NO: 53 for the light chain); DVD3754 (comprising SEQ ID NO: 54 for the heavy chain and SEQ ID NO: 55 for the light chain); DVD3755 (comprising SEQ ID NO: 56 for the heavy chain and SEQ ID NO: 57 for the light chain); DVD3756 (comprising SEQ ID NO: 58 for the heavy chain and SEQ ID NO: 59 for the light chain); DVD3757 (comprising SEQ ID NO: 60 for the heavy chain and SEQ ID NO: 61 for the light chain); DVD3758 (comprising SEQ ID NO: 62 for the heavy chain and SEQ ID NO: 63 for the light chain); DVD3759 (comprising SEQ ID NO: 64 for the heavy chain and SEQ ID NO: 65 for the light chain); DVD3760 (comprising SEQ ID NO: 66 for the heavy chain and SEQ ID NO: 67 for the light chain); DVD3761 (comprising SEQ ID NO: 68 for the heavy chain and SEQ ID NO:69 for the light chain); DVD3762 (comprising SEQ ID NO: 70 for the heavy chain and SEQ ID NO:71 for the light chain); DVD3764 (comprising SEQ ID NO: 72 for the heavy chain and SEQ ID NO:73 for the light chain); DVD3765 (comprising SEQ ID NO: 74 for the heavy chain and SEQ ID NO:75 for the light chain); DVD3766 (comprising SEQ ID NO: 76 for the heavy chain and SEQ ID NO:77 for the light chain); DVD3767 (comprising SEQ ID NO: 78 for the heavy chain and SEQ ID NO:79 for the light chain); DVD3769 (comprising SEQ ID NO: 80 for the heavy chain and SEQ ID NO:81 for the light chain); and DVD3770 (comprising SEQ ID NO: 82 for the heavy chain and SEQ ID NO:83 for the light chain).

In certain embodiments, the binding protein comprises a heavy chain and a light chain sequence for each of IgM and DNA, as shown in the Table 2 herein.

Any of the heavy chain, light chain, two chain, or four chain embodiments, can include at least one X1 linker comprising AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9), SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); GGGGSGGGGS (SEQ ID NO: 29); GGSGGGGSG (SEQ ID NO: 30); or G/S based sequences (e.g., G4S and G4S repeats; SEQ ID NO: 31). In certain embodiments, X1 is not a constant region, is not a CH region, or is not a CL region. In certain embodiments, X2 is an Fc region. In certain embodiments, X2 is a variant Fc region.

In certain embodiments, any of the heavy chain, light chain, two chain, or four chain embodiments can include at least one X1 linker comprising ASTKGP (SEQ ID NO: 21); ASTKGPSVFPLAP (SEQ ID NO: 22), TVAAP (SEQ ID NO: 13); and TVAAPSVFIFPP (SEQ ID NO: 14). In certain embodiments, the heavy chain comprises SEQ ID No: 21 and light chain comprises SEQ ID NO: 13. In certain embodiments, the heavy chain comprises SEQ ID No: 22 and light chain comprises SEQ ID NO: 14. In certain embodiments, the heavy chain comprises SEQ ID No: 21 and light chain comprises SEQ ID NO: 14. In certain embodiments, the heavy chain comprises SEQ ID No: 22 and light chain comprises SEQ ID NO: 13.

In certain embodiments, the Fc region, if present in the first polypeptide, is a native sequence Fc region or a variant sequence Fc region. In certain embodiments, the Fc region is an Fc region from an IgG1, an Fc region from an IgG2, an Fc region from an IgG3, an Fc region from an IgG4, an Fc region from an IgA, an Fc region from an IgM, an Fc region from an IgE, or an Fc region from an IgD.

In another aspect, the disclosure provides a method of making a binding protein that binds an immune cell receptor and/or an autoantigen. In certain embodiments, the method of making a binding protein that binds an immune cell receptor and/or an autoantigen comprises the steps of a) obtaining a first parent antibody, or antigen binding portion thereof, that binds an immune cell receptor; b) obtaining a second parent antibody, or antigen binding portion thereof, that binds an autoantigen; c) preparing construct(s) encoding any of the binding proteins described herein; and d) expressing the polypeptide chains, such that a binding protein that binds an immune cell receptor and/or an autoantigen is generated.

In certain embodiments, the first parent antibody or antigen binding portion thereof, and the second parent antibody or antigen binding portion thereof, are a mouse antibody, a human antibody, a CDR grafted antibody, a humanized antibody, and/or an affinity matured antibody.

In certain embodiments, the binding protein possesses at least one desired property exhibited by the first parent antibody or antigen binding portion thereof, or the second parent antibody or antigen binding portion thereof. Alternatively, the first parent antibody or antigen binding portion thereof and the second parent antibody or antigen binding portion thereof possess at least one desired property exhibited by the binding protein. In certain embodiments, the desired property is one or more antibody parameters. In certain embodiments, the antibody parameters are antigen specificity, affinity to antigen, potency, biological function, epitope recognition, stability, solubility, production efficiency, immunogenicity, pharmacokinetics, bioavailability, tissue cross reactivity, or orthologous antigen binding. In certain embodiments, the binding protein is multivalent. In certain embodiments, the binding protein is multispecific. The multivalent and or multispecific binding proteins described herein have desirable properties particularly from a therapeutic standpoint. For instance, the multivalent and or multispecific binding protein may (1) be internalized (and/or catabolized) faster than a bivalent antibody by a cell expressing an antigen to which the antibodies bind; (2) be an agonist binding protein; and/or (3) induce cell death and/or apoptosis of a cell expressing an antigen to which the multivalent binding protein is capable of binding. The “parent antibody”, which provides at least one antigen binding specificity of the multivalent and or multispecific binding protein, may be one that is internalized (and/or catabolized) by a cell expressing an antigen to which the antibody binds; and/or may be an agonist, cell death-inducing, and/or apoptosis-inducing antibody, and the multivalent and or multispecific binding protein as described herein may display improvement(s) in one or more of these properties. Moreover, the parent antibody may lack any one or more of these properties, but may acquire one or more of them when constructed as a multivalent binding protein as described herein. For example, different Fc mutants may prevent FcR, C′ binding, or extend half-life.

In certain embodiments, the binding protein has an on rate constant (Kₒₙ) to one or more targets of at least about 10²M⁻¹s⁻¹; at least about 10³M⁻¹s⁻¹; at least about 10⁴M⁻¹s⁻¹; at least about 10⁵M⁻¹s⁻¹; or at least about 10⁶M⁻¹s⁻¹, as measured by surface plasmon resonance. In certain embodiments, the binding protein has an on rate constant (Kₒₙ) to one or more targets from about 10²M⁻¹s⁻¹ to about 10³M⁻¹s⁻¹; from about 10³M⁻¹s⁻¹ to about 10⁴M⁻¹s⁻¹; from about 10⁴M⁻¹s⁻¹ to about 10⁵M⁻¹s⁻¹; or from about 10⁵M⁻¹s⁻¹ to about 10⁶M⁻¹s⁻¹, as measured by surface plasmon resonance.

In certain embodiments, the binding protein has an off rate constant (K_{off}) for one or more targets of at most about 10⁻³s⁻¹; at most about 10⁻⁴s⁻¹; at most about 10⁻⁵s⁻¹; or at most about 10⁻⁶s⁻¹, as measured by surface plasmon resonance. In certain embodiments, the binding protein has an off rate constant (K_{off}) to one or more targets of about 10⁻³s⁻¹ to about 10⁻⁴s⁻¹; of about 10⁻⁴s⁻¹ to about 10⁻⁵s⁻¹; or of about 10⁻⁵s⁻¹ to about 10⁻⁶s⁻¹, as measured by surface plasmon resonance.

In certain embodiments, the binding protein has a dissociation constant (K_{d}) to one or more targets of at most about 10⁻⁷M; at most about 10⁻⁸M; at most about 10⁻⁹M; at most about 10⁻¹⁹M; at most about 10⁻¹¹M; at most about 10⁻¹²M; or at most 10⁻¹³M. In certain embodiments, the binding protein has a dissociation constant (K_{d}) to its targets of about 10⁻⁷M to about 10⁻⁸M; of about 10⁻⁸M to about 10⁻⁹M; of about 10⁻⁹M to about 10⁻¹⁰ M; of about 10⁻¹⁰M to about 10⁻¹¹M; of about 10⁻¹¹M to about 10⁻¹²M; or of about 10⁻¹² to M about 10⁻¹³M, as measured by surface plasmon resonance.

In certain embodiments, the binding protein is a conjugate further comprising an agent. In certain embodiments, the agent can be an immunoadhesion molecule, an imaging agent, a therapeutic agent, or a cytotoxic agent.

In certain embodiments, the bispecific binding protein or binding protein conjugate is acid sensitive such that the binding protein is cleaved in an acidic environment. In certain embodiments, binding protein conjugate is acid sensitive such that the agent is released in an acidic environment.

In certain embodiments, the imaging agent is a radiolabel, an enzyme, a fluorescent label, a luminescent label, a bioluminescent label, a gold particle, a magnetic label, or biotin.

In certain embodiments, the radiolabel is ³H, ¹⁴C, ³⁵S, ⁹⁰Tc, ¹¹¹In, ¹²⁵I, ¹³¹I, ¹⁷⁷Lu, ¹⁶⁶Ho, or ¹⁵³Sm. In certain embodiments, the therapeutic or cytotoxic agent comprises an anti-metabolite, an alkylating agent, an antibiotic, a growth factor, a cytokine, an anti-angiogenic agent, an anti-mitotic agent, an anthracycline, toxin, or an apoptotic agent, or an immunosuppressive agent.

In certain embodiments, the binding protein comprises a biotin acceptor peptide sequence.

In certain embodiments, the binding protein is glycosylated. For example, the binding protein can comprise a human glycosylation pattern.

In another aspect, the disclosure provides isolated nucleic acids encoding any one of the binding proteins disclosed herein.

In another aspect, the disclosure provides vectors comprising any one of the isolated nucleic acids disclosed herein wherein the vector is pcDNA; pTT (Durocher et al. (2002) Nucleic Acids Res. 30(2); pTT3 (pTT with additional multiple cloning site; pEFBOS (Mizushima and Nagata (1990) Nucleic Acids Res. 18(17); pBV; pJV; pcDNA3.1 TOPO; pEF6 TOPO; pBOS; pHybE; or pBJ. In certain embodiments, the vector is a vector disclosed in U.S. Pat. No. 8,187,836. In certain embodiments, the vector is pCDNA 3.3 (Life Technologies).

In another aspect, the disclosure provides host cells transformed with the vectors disclosed herein. In certain embodiments, the host cell is a prokaryotic cell, for example, E. coli. In certain embodiments, the host cell is a eukaryotic cell, for example, a protist cell, an animal cell, a plant cell, or a fungal cell. In certain embodiments, the host cell is a mammalian cell including, but not limited to, CHO, COS, NS0, SP2, PER.C6, or a fungal cell, such as Saccharomyces cerevisiae, or an insect cell, such as Sf9. In certain embodiments, two or more binding proteins, e.g., with different specificities, are produced in a single recombinant host cell. For example, the expression of a mixture of antibodies has been called Oligoclonics™ (Merus B.V., The Netherlands) U.S. Pat. Nos. 7,262,028 and 7,429,486.

In another aspect, the disclosure provides a method of producing a bispecific binding protein of the invention comprising the step of culturing the host cells of the invention in culture medium under conditions sufficient to produce the bispecific binding protein.

In another aspect, the disclosure provides methods of producing the binding proteins disclosed herein comprising culturing any one of the host cells disclosed herein in a culture medium under conditions sufficient to produce the binding protein. In certain embodiments, 50%-75% of the binding protein produced by this method is a dual specific tetravalent binding protein. In certain embodiments, 75%-90% of the binding protein produced by this method is a dual specific tetravalent binding protein. In certain embodiments, 90%-95% of the binding protein produced is a dual specific tetravalent binding protein.

In another aspect, the disclosure provides a composition for the release of a binding protein wherein the composition comprises a crystallized binding protein, an ingredient, and at least one polymeric carrier. In certain embodiments, the polymeric carrier is poly (acrylic acid), a poly (cyanoacrylate), a poly (amino acid), a poly (anhydride), a poly (depsipeptide), a poly (ester), poly (lactic acid), poly (lactic-co-glycolic acid) or PLGA, poly (b-hydroxybutryate), poly (caprolactone), poly (dioxanone), poly (ethylene glycol), poly ((hydroxypropyl) methacrylamide, poly [(organo)phosphazene], a poly (ortho ester), poly (vinyl alcohol), poly (vinylpyrrolidone), a maleic anhydride-alkyl vinyl ether copolymer, a pluronic polyol, albumin, alginate, cellulose, a cellulose derivative, collagen, fibrin, gelatin, hyaluronic acid, an oligosaccharide, a glycaminoglycan, a sulfated polysaccharide, or blends and copolymers thereof. In certain embodiments, the ingredient is albumin, sucrose, trehalose, lactitol, gelatin, hydroxypropyl-β-cyclodextrin, methoxypolyethylene glycol, or polyethylene glycol.

In another aspect, the disclosure provides a method for treating a mammal comprising the step of administering to the mammal an effective amount of a composition disclosed herein.

In another aspect, the disclosure provides a pharmaceutical composition comprising a binding protein or binding protein conjugate disclosed herein and a pharmaceutically acceptable carrier. In certain embodiments, the pharmaceutical composition comprises at least one additional therapeutic agent for treating a disorder. For example, the additional agent may be a therapeutic agent, an imaging agent, a cytotoxic agent, an angiogenesis inhibitor (including but not limited to an anti-VEGF antibody or a VEGF-trap), a kinase inhibitor (including but not limited to a KDR and a TIE-2 inhibitor), a co-stimulation molecule blocker (including but not limited to anti-B7.1, anti-B7.2, CTLA4-Ig, anti-CD20), an adhesion molecule blocker (including but not limited to an anti-LFA-1 antibody, an anti-E/L selectin antibody, a small molecule inhibitor), an anti-cytokine antibody or functional fragment thereof (including but not limited to an anti-IL-18, an anti-TNF, and an anti-IL-6/cytokine receptor antibody), methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

In certain embodiments, the therapeutic agent is an inhibitor of B cell activation and/or an inhibitor of B cell proliferation and/or an inducer of B cell death.

In certain embodiments, the therapeutic agent can be an inhibitor B lymphocyte stimulator (BLys) such as, for example, belimumab, tabalumab, blisibimod or atacicept, or a combination thereof.

In another aspect, the disclosure provides a method for treating a human subject suffering from a disorder in which the target, or targets, capable of being bound by a binding protein disclosed herein is detrimental, comprising administering to the human subject a binding protein disclosed herein such that the activity of the target, or targets, in the human subject is inhibited and one or more symptoms is alleviated or treatment is achieved. The binding proteins provided herein can be used to treat humans suffering from autoimmune diseases such as, for example, those associated with TLR signaling. In certain embodiments, the binding proteins provided herein or antigen binding portions thereof, are used to treat inflammation, asthma, allergies, allergic lung disease, allergic rhinitis, atopic dermatitis, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, infectious diseases caused by HIV, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, occular surface inflammatory diseases, or Lyme disease.

In another aspect, the disclosure provides methods of determining a patient's reactivity to a therapeutic agent that is capable of modulating, e.g., inhibiting or inducing, the activity of a TLR, the method comprising the steps of (a) obtaining a cell sample from a patient; (b) treating a first portion of the cell sample with a therapeutic agent in the presence of the bispecific binding protein that binds a TLR-activating autoantigen and an immune cell receptor; (c) treating a second portion of the cell sample with the therapeutic agent in the absence of the bispecific binding protein; and (d) measuring cell proliferation and/or cell death of the cell samples of steps (b) and (c); wherein a difference in cell proliferation and/or cell death in the two cell samples is indicative of the patient's reactivity to the therapeutic agent. In certain embodiments, the patient is in need of a TLR inhibitor. In certain embodiments, the patient is in need of a TLR inducer. The method can be used to determine the patient's inclusion in, or eligibility for, a clinical trial for the therapeutic agent, e.g., to assess the efficacy of the therapeutic agent. The patient may be suspected of having an autoimmune disease that comprises activation of a TLR, e.g., TLR7 or TLR9, such as systemic lupus erythematosus (SLE), lupus nephritis, discoid lupus, neonatal lupus, Sjogren's disease, dermatomyostitis and systemic sclerosis. In certain embodiments, the cell sample comprises a B cell.

In another aspect, the disclosure provides methods of identifying a BCR inhibitor or activator, comprising the steps of: (a) treating TLR9 responsive cells with a candidate molecule in the presence or absence of a binding protein that binds a TLR-activating autoantigen and an immune cell receptor; and (b) measuring proliferation and/or death of the TLR9 responsive cell compared to a control, wherein a difference in proliferation and/or death in the TLR9 responsive cell compared to the control is indicative of a patient's reactivity to the candidate molecule.

In another aspect, the disclosure provides for a method of administering a pharmaceutical composition comprising a bispecific binding protein that binds a TLR-activating autoantigen and an immune cell receptor to a subject in need thereof.

In another aspect, the disclosure provides a method for activating or inhibiting TLR9 responsive cells in a patient in need of TLR9 activation or TLR9 inhibition, respectively, the method comprising the step of administering the pharmaceutical composition of the invention to a patient in need thereof.

In another aspect, the disclosure provides methods for treating a patient in need of TLR9 activation or TLR9 inhibition, the method comprising the steps of (a) obtaining a cell sample comprising TLR9 responsive cells from the patient; (b) treating the patient's TLR9 responsive cells with a pharmaceutical composition comprising a bispecific binding protein that binds a TLR-activating autoantigen and an immune cell receptor; and (c) reintroducing the treated cells into the patient.

In another aspect, the disclosure provides for a method of identifying a BCR inhibitor comprising the steps of (a) treating a TLR7 responsive cell with a candidate molecule in the presence or absence of a binding protein that binds a TLR-activating autoantigen and an immune cell receptor; and (b) measuring proliferation and/or death of the TLR7 responsive cell compared to a control, wherein the a difference in proliferation and/or death in the TLR7 responsive cell compared to the control is indicative that the candidate molecule is a BCR inhibitor.

In another aspect, the disclosure provides methods of activating TLR7 responsive cells in a subject in need of TLR7 activation comprising administering a pharmaceutical preparation comprising a bispecific binding protein that binds a TLR-activating autoantigen and an immune cell receptor to a subject in need thereof.

In another aspect, the disclosure provides for a method of activating TLR7 responsive cells in a subject in need of TLR7 activation comprising treating a subject's TLR7 responsive cells with a pharmaceutical preparation comprising a bispecific binding protein that binds a TLR-activating autoantigen and an immune cell receptor and reintroducing the treated cells into the subject.

In another aspect, the disclosure provides methods of identifying an inhibitor or stimulator of TLR signaling, the method comprising the steps of a) combining a test agent, a B cell, and the bispecific binding protein that binds a TLR-activating autoantigen and an immune cell receptor under conditions suitable for detecting a bispecific binding protein-induced response in the B cell; and b) determining the ability of the test agent to inhibit or stimulate, respectively, the bispecific binding protein-induced response in the B cell, wherein an inhibition of the bispecific binding protein-induced response is indicative that the test agent is an inhibitor or wherein an stimulation of the bispecific binding protein-induced response is indicative that the test agent is a stimulator of TLR signaling.

In certain embodiments, the TLR can be TLR 7, TLR9, or TLR3. The bispecific binding protein-induced response can result in cell proliferation and/or cell death.

In another aspect, the disclosure provides a kit for assaying a test sample for an immune cell receptor and an autoantigen, or fragment thereof. The kit comprises at least one component for assaying the test sample for an immune cell receptor and an TLR-activating autoantigen, or fragment thereof, and instructions for assaying the test sample for the immune cell receptor and the autoantigen, wherein the at least one component includes at least one composition comprising the binding protein, wherein the binding protein is optionally detectably labeled.

### BRIEF DESCRIPTION OF THE DRAWINGS

FIG. 1 is a diagram of a DVD-Ig binding protein that bind to IgM and nucleic acid (DNA or RNA), in two orientations.

FIG. 2 is a diagram of a DVD-Ig binding protein bound to nucleic acid and to a B cell receptor (BCR) on a B cell.

FIG. 3 show data from mouse B cells that are treated with (a) media, with and without B Lymphocyte Stimulator (BLys); (b) Toll Like Receptor 9 (TLR9) ligand 1826, with and without BLys; (c) PL2-3, an anti-chromatin IgG2a, with and without BLys; (d) bispecific anti-IgM and anti-DNA DVD-Ig binding protein DVD3759, with and without BLys. Cell proliferation was quantified by carboxyfluorescein diacetate, succinimidyl ester (CFSE) dilution and cell death by Sytox Blue binding.

FIG. 4 shows data from B cells isolated from wild-type (WT), IRAK2 knock out (IRAK2 KO), and IRAK4 kinase-dead knock-in mice (IRAK4 KI) that are treated for 60-72 hours, in duplicate, with (a) bispecific anti-IgM and anti-DNA DVD-Ig binding protein DVD3759, with and without BLys, or (b) Toll Like Receptor 9 (TLR9) ligand 1826, with and without BLys. Cell proliferation was quantified by CFSE dilution and cell death by TO-PRO-3 binding.

FIG. 5 shows the results of experiments to examine the proliferation of primary human B cells in response to stimulation through BCR and TLR-9 by the indicated DVD-Ig proteins.

FIG. 6 shows the results of experiments to examine the proliferation of primary human B cells in response to 3764 DVD-Ig protein or the CpG oligonucleotide ODN2006 (in both the presence and absence of a TLR9 inhibitor).

FIG. 7 shows (A) DKO ANA immunofluorescent staining patterns from DNase Het, DKO and TKO mice at 25 weeks and 40 weeks of age on HEp-2 coated slides; and (B) a summary of DKO ANA staining patterns from mice at early stages of the disease process. “Nucleolar” indicates a prominent nucleolar pattern, “speckled nuclear” refers to a non-nucleolar speckled pattern, “cytoplasmic” refers to a diffuse cytoplasmic stain, and “other” includes antibodies that appear to be directed at proliferating cells.

FIG. 8 shows (A) a schematic diagram of bifunctional DVD-Ig™ binding proteins; (B) a graph of IgM-binding ELISA of representative DVD-Ig™ binding proteins, with anti-IgM domain as V1 (DVD3756, blue), or with the anti-IgM domain as V2 (DVD3751, red; DVD3754, green), compared to the original anti-IgM antibody; (C) ANA staining patterns of the anti-DNA mAb compared to the DVD-Ig™ binding proteins depicted in (B); and (D) a composite plot of EC50 and ANA score with capacity of each DVD-Ig™ binding proteins to activate B cells, as determined by ³H-thymidine incorporation, indicated by the size of the circle; proliferation index, large circle >20-fold; medium circle 10-20 fold; small circle <10-fold). The color of the circle corresponds to the DVD-Ig™ binding proteins depicted in B and C, additional DVD-Ig™ binding proteins depicted as open black circles, and original mAbs indicated by filled black circles.

FIG. 9 shows (A) FACS analyses of B220+ B cells from RF (AM14 WT, AM14 Tlr9^{-/-}) or non-Tg (BALB/c WT, BALB/c Tlr9^{-/-}) mice isolated with B220-specific magnetic beads, labeled with CFSE and stimulated with anti-DNA mAb, ODN1826, or DVD3754 for 72 hours and compared to medium control; and (B) 3H-thymidine incorporation of B cells from the mice in (A) stimulated with the DVD3754 or anti-DNA mAb for 24 hours and compared to medium control. The data represent the average of 3 separate experiments+/−the SEM.

FIG. 10 shows (A) a graph of spleen weights from DNase Het, DKO and TKO mice at 10 weeks of age, where each dot represents 1 mouse (n=12 for all groups); (B) spleen cells stained for B220 and AA4.1 to enumerate the total number of splenic B cells (Y-axis) and the % of mature B cells, where the % mature B cells are indicated by the grey portion of the bar and inserted number; (C) ³H-thymidine incorporation of B220-purified B cells from the DNase Het, DKO and TKO mice stimulated with anti-IgM, ODN 1826 or DVD3754 for 24 hours. Results in (B) and (C) are from 3 separate experiments include 8 mice/group and are summarized as CPM+/−SEM.

### DETAILED DESCRIPTION

Multivalent and/or multispecific binding proteins capable of binding immune cell receptors and autoantigens are provided. Bispecific binding proteins, e.g., dual variable domain immunoglobulin (DVD-Ig™) binding proteins, and pharmaceutical compositions thereof, as well as nucleic acids, recombinant expression vectors and host cells for making such bispecific binding proteins are also provided. Methods of using the bispecific binding proteins to detect specific antigens, either in vitro or in vivo are also provided.

### I. DEFINITIONS

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Generally, nomenclatures used in connection with cell and tissue culture, molecular biology, immunology, microbiology, genetics and protein and nucleic acid chemistry and hybridization described herein are those well known in the art. The methods and techniques provided herein are generally performed according to conventional methods well known in the art and as described in the references that are cited and discussed throughout the specification unless otherwise indicated. Enzymatic reactions and purification techniques are performed according to manufacturer's specifications, as commonly accomplished in the art or as otherwise described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

Select terms are defined below so that the disclosure may be more readily understood.

The term “subject”, as used herein, generally refers to an animal, such as a mammal. A subject can therefore refer to, for example, dogs, cats, horses, cows, pigs, guinea pigs, and the like. Preferably the subject is a human. When the subject is a human, the subject may be referred to herein as a “patient”. The terms “treat,” “treating” or “treatment” of a disease of a subject refers to any improvement in one or more clinical symptoms of the disease.

The terms “Toll-Like Receptor” and “TLR” refer to a member of the Toll-like family of receptors. Currently, ten mammalian homologues have been identified, called TLR1 through TLR10. TLRs can activate downstream immune response genes through signaling cascades that include the adaptor protein MyD88 (Mussio et al. (1997) Science 278:1612; Wesche et al. (1997) Immunity 7(6):837-47). In addition to microbial particles, mammalian TLRs can also recognize certain self antigens, in particular cytoplasmic components that are released from cells as a result of cell death (Akira et al. (2000) Nature Immunol. 2: 675-680). The term “TLR” includes an intact Toll-like receptor, for example, a receptor that has been described in the Online Mendelian Inheritance in Man under access numbers *601194 TOLL-LIKE RECEPTOR 1, TLR1; *603028 TOLL-LIKE RECEPTOR 2, TLR2; *603029 TOLL-LIKE RECEPTOR 3, TLR3; *603030 TOLL-LIKE RECEPTOR 4, TLR4; *603031 TOLL-LIKE RECEPTOR 5, TLR5; *605403 TOLL-LIKE RECEPTOR 6, TLR6; *300365 TOLL-LIKE RECEPTOR 7, TLR7; *300366 TOLL-LIKE RECEPTOR 8, TLR8; *605474 TOLL-LIKE RECEPTOR 9; TLR9; and *606270 TOLL-LIKE RECEPTOR 10; TLR10 or a fragment or functional fragment thereof such as, for example, a soluble form of the Toll-like receptor, i.e., where the membrane binding domain has been deleted or altered. TLRs include a MyD88 binding or interacting fragment of the Toll-like receptor or a homolog of the Toll-like receptor capable of binding to or interacting with MyD88. In certain embodiments, the TLR is an endosomal TLR, e.g., TLR7 or TLR 9, or a fragment or functional fragment, or homologue thereof. In certain embodiments, the cytoplasmic domain of the TLR is not present.

The term “immunoadhesion molecule” refers to an antibody-like molecule that combines the binding domain of a non-antibody polypeptide with the effector functions of an antibody or an antibody constant domain.

The terms “B lymphocyte stimulator” and “BLyS” refer to the human tumor necrosis factor (TNF) superfamily cytokine that is encoded by the TNFSF13B gene, also referred to as “B-cell activating factor” (BAFF). Exemplary BLyS proteins are set forth in Genbank accession numbers GI:5730097 and GI: 224548983.

The term “bispecific binding protein” refers to an polypeptide having at least two distinct antigen binding sites, such that it can simultaneously bind to at least two targets and have specificity for two different targets, i.e., either two different antigens or two different epitopes on the same antigen, with the proviso that the antigen binding sites of the bispecific binding protein are not antibody Fc regions. The two targets may be located on the same molecule, e.g., different epitopes on the same antigen, or may be located on separate molecules, e.g., on two different cells or on a cell and a on soluble antigen. Bispecific binding proteins include bispecific antibodies but also include fusion proteins comprising known antibody components as well as a variety of other formats, including format a DVD-Ig™ molecule, a BiTe® molecule, a DART® molecule, a DuoBody™ molecule, a scFv/diabody-IgG molecule, a cross-over multispecific (e.g., bispecific) molecule, a 2-in-1 bispecific molecule, a knob-in-hole multispecific (e.g., bispecific) molecule, a CovXBody molecule, an affibody molecule, a scFV/diabody-CH2/CH3 bispecific molecule, a IgG-non-Ig protein scaffold-based multispecific (e.g., bispecific) molecule, and a scFV/diabody linked to normal human protein like human serum albumin-bispecific molecule. Examples of different formats of bispecific binding proteins can be found in U.S. Pat. No. 7,612,181.

The term “antibody” refers to an immunoglobulin (Ig) molecule, which generally comprises of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In certain embodiments of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: ER1, CDR1, ER2, CDR2, ER3, CDR3, and ER4. Immunoglobulin molecules can be of any type (e.g., IgG, IgE, IgM, IgD, IgA and IgY), class (e.g., IgG1, IgG2, IgG3, IgG4, IgA1 and IgA2), or subclass. Variability exists in the endogenous antibodies between the species.

The term “bispecific antibody” refers to an antibody that binds one antigen (or epitope) on one of its two binding arms (one pair of HC/LC), and binds a different antigen (or epitope) on its second binding arm (a different pair of HC/LC). A bispecific antibody has two distinct antigen binding arms (in both specificity and CDR sequences), and is monovalent for each antigen to which it binds. Bispecific antibodies have been produced using the quadroma technology (Milstein and Cuello (1983) Nature 305(5934):537-40) based on the somatic fusion of two different hybridoma cell lines expressing murine monoclonal antibodies with the desired specificities of the bispecific antibody. Because of the random pairing of two different Ig heavy and light chains within the resulting hybrid-hybridoma (or quadroma) cell line, up to ten different immunogloblin species are generated, of which only one is the functional bispecific antibody. The presence of mispaired by-products, and significantly reduced production yields, means sophisticated purification procedures are required.

Bispecific antibodies can also be produced by chemical conjugation of two different mAbs (Staerz et al. (1985) Nature 314(6012):628-31), however this approach does not yield homogeneous preparation. Other approaches have used chemical conjugation of two different monoclonal antibodies or smaller antibody fragments (Brennan et al. (1985) Science 229(4708):81-3). Another method for making a bispecific antibody is the coupling of two parental antibodies with a hetero-bifunctional crosslinker, but the resulting preparations of bispecific antibodies suffer from significant molecular heterogeneity because reaction of the crosslinker with the parental antibodies is not site-directed. To obtain more homogeneous preparations of bispecific antibodies two different Fab fragments have been chemically crosslinked at their hinge cysteine residues in a site-directed manner (Glennie et al. (1987) J. Immunol. 139(7):2367-75). However, this method results in Fab′2 fragments, not full IgG molecules.

A wide variety of other recombinant bispecific antibody formats have been developed (Kriangkum et al. (2001) Biomol. Engin. 18(2):3140) including tandem single-chain Fv molecules and diabodies, and various derivatives there of, which are the most widely used formats for the construction of recombinant bispecific antibodies. Routinely, construction of these molecules starts from two single-chain Fv (scFv) fragments that recognize different antigens (Economides et al. (2003) Nature Med. 9(1):47-52). Tandem scFv molecules (taFv) are made by connecting the two scFv molecules with an additional peptide linker. The two scFv fragments present in these tandem scFv molecules form separate folding entities. Various linkers can be used to connect the two scFv fragments and linkers with a length of up to 63 residues are most effective (Nakanishi et al. (2001) Ann. Rev. Immunol. 19:423-74). Although the parental scFv fragments can normally be expressed in soluble form in bacteria, tandem scFv molecules form insoluble aggregates in bacteria. Hence, refolding protocols or the use of mammalian expression systems are routinely applied to produce soluble tandem scFv molecules. In vivo expression by transgenic rabbits and cattle of a tandem scFv directed against CD28 and a melanoma-associated proteoglycan has been reported (Gracie et al. (1999) J. Clin. Invest. 104(10):1393-401). In this construct, the two scFv molecules were connected by a CH1 linker and serum concentrations of up to 100 mg/L of the bispecific antibody were found. Various strategies including variations of the domain order or using middle linkers with varying length or flexibility were employed to allow soluble expression in bacteria. Others have also reported expression of soluble tandem scFv molecules in bacteria (Leung et al. (2000) J. Immunol. 164(12): 6495-502; Ito et al. (2003) J. Immunol. 170(9):4802-9; Karni et al. (2002) J. Neuroimmunol. 125(1-2):134-40) using either a very short Ala3 linker or long glycine/serine-rich linkers. Phage display of a tandem scFv repertoire containing randomized middle linkers with a length of 3 or 6 residues can be employed to enrich for those molecules that are produced in soluble and active form in bacteria. This approach resulted in the isolation of a preferred tandem scFv molecule with a 6 amino acid residue linker (Arndt and Krauss (2003) Methods Mol. Biol. 207:305-21). Although it is unclear whether this linker sequence represents a general solution to the soluble expression of tandem scFv molecules, this study demonstrated that phage display of tandem scFv molecules in combination with directed mutagenesis can enrich for these molecules, which can be expressed in bacteria in an active form.

Bispecific diabodies (Db) utilize the diabody format for expression. Diabodies are produced from scFv fragments by reducing the length of the linker connecting the VH and VL domain to approximately 5 residues (Peipp and Valerius (2002) Biochem. Soc. Trans. 30(4):507-11). This reduction of linker size facilitates dimerization of two polypeptide chains by crossover pairing of the VH and VL domains Bispecific diabodies are produced by expressing two polypeptide chains with either the structure VHA-VLB and VHB-VLA (VH-VL configuration) or the structure VLA-VHB and VLB-VHA (VL-VH configuration) within the same cell. A variety of different bispecific diabodies have been produced and most of them can be expressed in soluble form in bacteria. However, the orientation of the variable domains can influence expression and formation of active binding sites (Mack et al. (1005) Proc. Natl. Acad. Sci. USA 92(15):7021-5). Nevertheless, soluble expression in bacteria represents an important advantage over tandem scFv molecules. However, since two different polypeptide chains are expressed within a single cell, inactive homodimers can be produced together with active heterodimers, which necessitates additional purification steps in order to obtain homogenous preparations. Another approach to force the generation of bispecific diabodies is the production of knob-into-hole diabodies (Holliger et al. (1993) Proc. Natl. Acad. Sci. USA 90(14):6444-8.18). This approach was demonstrated for a bispecific diabody directed against HER2 and CD3. A large knob was introduced in the VH domain by exchanging Va137 with Phe and Leu45 with Trp and a complementary hole was produced in the VL domain by mutating Phe98 to Met and Tyr87 to Ala, either in the anti-HER2 or the anti-CD3 variable domains. Using this approach the production of bispecific diabodies could be increased from 72% by the parental diabody to over 90% by the knob-into-hole diabody. Importantly, production yields only slightly decreased as a result of these mutations. However, a reduction in antigen binding activity was observed for several constructs. Thus, this rather elaborate approach requires the analysis of various constructs in order to identify those mutations that produce heterodimeric molecule with unaltered binding activity. In addition, such approach requires mutational modification of the immunoglobulin sequence at the constant region, thus creating non-native and non-natural forms of the antibody sequence, which may result in increased immunogenicity, poor in vivo stability, and undesirable pharmacokinetics.

Single-chain diabodies (scDb) represent an alternative strategy for improving the formation of bispecific diabody-like molecules (Holliger and Winter (1997) Cancer Immunol. Immunother. 45(3-4):128-30; Wu et al. (1996) Immunotechnol. 2(1):21-36). Bispecific single-chain diabodies are produced by connecting the two diabody-forming polypeptide chains with an additional middle linker of about 15 amino acid residues. Consequently, all molecules with a molecular weight corresponding to monomeric single-chain diabodies (50-60 kDa) are bispecific. Several studies have demonstrated that bispecific single chain diabodies are expressed in bacteria in soluble and active form with the majority of purified molecules present as monomers (Holliger and Winter (1997) Cancer Immunol. Immunother. 45(34):128-30; Wu et al. (1996) Immunotechnol. 2(1):21-36; Pluckthun and Pack (1997) Immunotechnol. 3(2):83-105; Ridgway et al. (1996) Protein Engin. 9(7):617-21). Single-chain diabodies therefor combine the advantages of tandem scFvs (all monomers are bispecific) and diabodies (soluble expression in bacteria).

Diabodies have also been fused to Fc to generate more Ig-like molecules, named di-diabody (Lu et al. (2004) J. Biol. Chem. 279(4):2856-65). In addition, multivalent antibody constructs comprising two Fab repeats in the heavy chain of an IgG and capable of binding four antigen molecules have been described (PCT Publication No. WO 0177342; Miller et al. (2003) J. Immunol. 170(9):4854-61).

The terms “dual variable domain binding protein” and “dual variable domain immunoglobulin” refer to a binding protein that has two variable domains in each of its two binding arms (e.g., a pair of HC/LC), each of which is able to bind to an antigen. In certain embodiments, each variable domain binds different antigens or epitopes. In certain embodiments, each variable domain binds the same antigen or epitope. In certain embodiments, a dual variable domain binding protein has two identical antigen binding arms, with identical specificity and identical CDR sequences, and is bivalent for each antigen to which it binds. In certain embodiments, the dual variable domain binding proteins may be monospecific, i.e., capable of binding one antigen or multispecific, i.e., capable of binding two or more antigens. Dual variable domain binding proteins comprising two heavy chain dual variable domain polypeptides and two light chain dual variable domain polypeptides are referred to as a DVD-Ig™ protein. In certain embodiments, each half of a four chain dual variable domain binding protein comprises a heavy chain dual variable domain polypeptide, and a light chain dual variable domain polypeptide, and two antigen binding sites. In certain embodiments, each binding site comprises a heavy chain variable domain and a light chain variable domain with a total of 6 CDRs involved in antigen binding per antigen binding site.

The term “anti-idiotypic antibody” refers to an antibody raised against the amino acid sequence of the antigen combining site of another antibody. Anti-idiotypic antibodies may be administered to enhance an immune response against an antigen.

The term “anti-allotypic antibody” refers to an antibody raised against the amino acid sequence of constant region of another antibody.

The term “biological activity” refers to one or more biological properties of a molecule (whether present naturally as found in vivo, or provided or enabled by recombinant means). Biological properties include, but are not limited to, binding a receptor, inducing cell proliferation or other cellular function, inhibiting cell growth or other cellular function, inducing cytokine production or activity, activating a signal transduction cascade, inducing apoptosis, and enzymatic activity.

The term “neutralizing” refers to counteracting the biological activity of an antigen, e.g., a binding protein may neutralize an antigen when it specifically binds to the antigen. In certain embodiments, the neutralizing binding protein binds to an antigen (e.g., a cytokine) and reduces its biological activity by at least about 20%, 40%, 60%, 80%, 85%, 90%, 95%, or more.

The term “specificity” refers to the ability of a binding protein to selectively bind an antigen.

The term, “affinity” refers the strength of the interaction between a binding protein and an antigen, and is determined by the sequence of the CDRs of the binding protein as well as by the nature of the binding protein and the antigen, such as their size, shape, and/or charge. Binding proteins may be selected for affinities that provide desired therapeutic end-points while minimizing negative side-effects Affinity may be measured using methods known to one skilled in the art (US 20090311253).

The term “potency” refers to the ability of a binding protein to achieve a desired effect, and is a measurement of its therapeutic efficacy. Potency may be assessed using methods known to one skilled in the art (US Patent Appl. No. 20090311253).

The term “biological function” refers the specific in vitro or in vivo actions of a binding protein. Binding proteins may target several classes of antigens and achieve desired therapeutic outcomes through multiple mechanisms of action. Binding proteins may target soluble proteins, cell surface antigens, as well as extracellular protein deposits. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve distinct functions in a single binding protein molecule. The in vitro assays and in vivo models used to assess biological function are known to one skilled in the art (US Patent Appl. No. 20090311253).

The terms “label” and “detectable label” mean a moiety attached to a member of a specific binding pair, such as an antibody or its analyte to render a reaction (e.g., binding) between the members of the specific binding pair, detectable. The labeled member of the specific binding pair is referred to as “detectably labeled.” Thus, the term “labeled binding protein” refers to a protein with a label incorporated that provides for the identification of the binding protein. In certain embodiments, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods) or immunogold. Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho or ¹⁵³Sm); chromogens, fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “binding protein conjugate” refers to a binding protein, such as an antibody, that is chemically linked to a chemical or biological moiety, such as a therapeutic or cytotoxic agent. The term “agent” includes a chemical compound, a mixture of chemical compounds, a biological macromolecule, or an extract made from biological materials. In certain embodiments, the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, for example, a conjugated antibody may be a detectably labeled antibody used as the detection antibody. When employed as a therapy, a conjugated binding protein may release the agent in a particular body or cellular compartment, e.g., in response to a change in the acidic environment.

The term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. The binding proteins described herein were constructed using the vector pCDNA 3.3 (Life Technologies). A group of pHybE vectors (US Patent Publication No. 20120237976) are also commonly used for parental antibody and bispecific binding protein cloning. V1, derived from pJP183; pHybE-hCg1,z,non-a V2, is used for cloning of antibody and bispecific binding protein heavy chains with a wildtype constant region. V2, derived from pJP191; pHybE-hCk V3, is used for cloning of antibody and bispecific binding protein light chains with a kappa constant region. V3, derived from pJP192; pHybE-hCl V2, is used for cloning of antibody and bispecific binding protein light chains with a lambda constant region. V4, built with a lambda signal peptide and a kappa constant region, is used for cloning of bispecific binding protein light chains with a lambda-kappa hybrid V domain. V5, built with a kappa signal peptide and a lambda constant region, is used for cloning of bispecific binding protein light chains with a kappa-lambda hybrid V domain. V7, derived from pJP183; pHybE-hCg1,z,non-a V2, is used for cloning of antibody and bispecific binding protein heavy chains with a (234,235 AA) mutant constant region.

The terms “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In certain embodiments, host cells include prokaryotic and eukaryotic cells. In certain embodiments, eukaryotic cells include protist, fungal, plant and animal cells. In certain embodiments, host cells include but are not limited to the prokaryotic cell line E. Coli; mammalian cell lines CHO, HEK 293, COS, NS0, SP2 and PER.C6; the insect cell line Sf9; and the fungal cell Saccharomyces cerevisiae.

The term “transfection” encompasses a variety of techniques commonly used for the introduction of exogenous nucleic acid (e.g., DNA) into a host cell, e.g., electroporation, calcium-phosphate precipitation, DEAE-dextran transfection and the like.

The term “cytokine” refers to a protein released by one cell population that acts on another cell population as an intercellular mediator. The term “cytokine” includes proteins from natural sources or from recombinant cell culture and biologically active equivalents of the native cytokines.

The term “biological sample” or “test sample” or “cell sample” means a quantity of a substance from a living thing or formerly living thing. Such substances include, but are not limited to, blood (e.g., whole blood), plasma, serum, urine, amniotic fluid, synovial fluid, endothelial cells, leukocytes, lymphocytes, monocytes, other cells, organs, tissues, bone marrow, lymph nodes and spleen.

The term “component” refers to an element of a composition. In relation to a diagnostic kit, for example, a component may be a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample. Thus, a “component” can include a polypeptide or other analyte as above, that is immobilized on a solid support, such as by binding to an anti-analyte (e.g., anti-polypeptide) antibody. Some components can be in solution or lyophilized for reconstitution for use in an assay.

The term “control” refers to a composition known to not contain an analyte or test substance (“negative control”) or to contain an analyte or test substance (“positive control”). A positive control can comprise a known concentration of an analyte or test substance. A “positive control” can be used to establish assay performance characteristics and is a useful indicator of the integrity of reagents (e.g., analytes or test substances). “Control,” “positive control,” and “calibrator” may also be used interchangeably herein to refer to a composition comprising a known concentration of an analyte or test substance.

The term “Fc region” defines the C-terminal region of an immunoglobulin heavy chain, which may be detached from the variable region of the immunoglobulin by papain digestion of an intact immunoglobulin. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary, or even deleterious, depending on the therapeutic objectives.

The term “antigen binding portion” or “antigen binding site” or “target binding site” of a binding protein means one or more fragments of a binding protein (e.g., an antibody or receptor), such as an immunoglobulin variable domain (e.g., VH or VL), that retain the ability to specifically bind to an antigen or target. The antigen binding portion of a binding protein can be performed by fragments of a full-length antibody, as well as bispecific, dual specific, or multi-specific formats; specifically binding to two or more antigens. Examples of binding fragments encompassed within the term “antigen binding portion” of an binding protein include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which comprises a single variable domain; and (vi) an isolated complementarity determining region (CDR). Furthermore, although the VH and VL of the Fv, which are encoded by separate genes, can be joined using recombinant methods by a synthetic linker that enables them to be made as a single protein chain in which the VH and VL regions pair to form monovalent molecules (known as single chain Fv (scFv). Such scFvs are also encompassed within the term “antigen binding portion” as are other forms of single chain antibodies, such as diabodies and “linear antibodies” comprising a pair of tandem Fv segments (VH-CH1-VH-CH1) which, together with complementary light chain polypeptides, form a pair of antigen binding sites. Not every amino acid of an antigen binding portion may bind to an antigen. For example, variable domains of an antibody comprise both complementarity determining regions (CDRs) and framework regions (FRs).

The term “multivalent binding protein” means a binding protein comprising two or more antigen binding sites. In certain embodiments, the multivalent binding protein is engineered to have three or more antigen binding sites, and is not a naturally occurring antibody. The term “multispecific binding protein” refers to a binding protein comprising two or more antigen binding sites capable of binding two or more targets, of which at least two targets are different. In certain embodiments, the bispecific binding proteins provided herein comprise two or more antigen binding sites and are tetravalent or multivalent binding proteins.

The term “linker” means an amino acid residue or a polypeptide comprising two or more amino acid residues joined by peptide bonds that are used to link two polypeptides (e.g., two VH or two VL domains) Such linker polypeptides are well known in the art (e.g., Holliger et al. (1993) Proc. Natl. Acad. Sci. USA 90:6444-6448; Poljak et al. (1994) Structure 2:1121-1123).

The term “CDR” means a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1971) Ann NY Acad. Sci. 190:382-391; Kabat et al. (1987) Sequences of Proteins of Immunological Interest, Fourth Edition. US Govt. Printing Off. No. 165-492; Kabat et al. (1991) Sequences of Proteins of Immunological Interest, Fifth Edition. NIH Publication No. 91-3242) not only provides an unambiguous residue numbering system applicable to any variable region of an antibody, but also provides precise residue boundaries defining the three CDRs. These CDRs may be referred to as Kabat CDRs. The terms “Kabat numbering”, “Kabat definitions” and “Kabat labeling” are used interchangeably herein to refer to a system of numbering amino acid residues that are more variable (e.g., hypervariable) than other amino acid residues in the heavy and light chain variable regions of an antibody. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems.

The term “surface plasmon resonance” means an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jonsson et al. (1993) Ann. Biol. Clin. 51:19-26. The term “Kₒₙ” means the on rate constant for association, or “association rate constant”, of a binding protein (e.g., an antibody or bispecific binding protein) to an antigen to form a binding protein/antigen complex. This value indicating the binding rate of a binding protein to its target antigen or the rate of complex formation between a binding protein, e.g., an antibody, and antigen also is shown by the equation below:

Antibody (“Ab”)+Antigen (“Ag”)→Ab−Ag

The term “K_{off}” means the off rate constant for dissociation, or “dissociation rate constant”, of a binding protein (e.g., an antibody or bispecific binding protein) from the binding protein/antigen complex. This value indicates the dissociation rate of a binding protein, e.g., an antibody, from its target antigen or separation of Ab−Ag complex over time into free antibody and antigen as shown by the equation below:

Ab+Ag←Ab−Ag

The terms “K_{d}” and “equilibrium dissociation constant” mean the value obtained in a titration measurement at equilibrium, or by dividing the dissociation rate constant (K_{off} by the association rate constant (Kₒₙ). The association rate constant, the dissociation rate constant, and the equilibrium dissociation constant are used to represent the binding affinity of a binding protein (e.g., an antibody or bispecific binding protein) to an antigen. Methods for determining association and dissociation rate constants are well known in the art. Fluorescence-based techniques offer high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can be used.

The term “variant” means a polypeptide that differs from a given polypeptide in amino acid sequence by the addition (e.g., insertion), deletion, or conservative substitution of amino acids, but that retains the biological activity of the given polypeptide (e.g., a variant anti-IgM antibody can compete with anti-IgM antibody for binding to IgM). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (e.g., Kyte et al. (1982) J. Mol. Biol. 157: 105-132). The hydropathic index of an amino acid is based on a consideration of its hydrophobicity and charge. It is known in the art that amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptide or fragment thereof that has been differentially processed, such as by proteolysis, phosphorylation, or other post-translational modification, yet retains its biological activity or antigen reactivity, e.g., the ability to bind to IgM or DNA. The term “variant” encompasses fragments of a variant unless otherwise defined. A variant may be 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% identical to the wildtype sequence.

The term “TLR signaling autoantigen” refers to an immunogenic antigen or epitope that is endogenous to an individual's physiology and that signals a TLR response. In certain embodiments, the TLR signaling autoantigen activates TLR-7 or TLR-9. In certain embodiments, the TLR signaling autoantigen is a self protein or protein complex that comprises DNA and/or RNA.

The term “TLR activating autoantigen” refers to an immunogenic antigen or epitope that is endogenous to an individual's physiology and that activates a TLR response. In certain embodiments, the TLR activating autoantigen activates TLR-7 or TLR-9. In certain embodiments, the TLR activating autoantigen is a self protein or protein complex that comprises DNA and/or RNA.

The term “TLR inhibiting autoantigen” refers to an immunogenic antigen or epitope that is endogenous to an individual's physiology and that inhibits a TLR response.

The term “autoimmune disease” can include, but is not limited to, acute disseminated encephalomyelitis, Addison's disease, agammaglobulinemia, alopecia greata, amyotrophic lateral sclerosis (also lou gehrig's disease; motor neuron disease), ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria autoimmune uveitis, balo disease/balo concentric sclerosis, Basedow's disease, Behçet's disease, Berger's disease Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, crest syndrome, Crohn's disease (one of two types of idiopathic inflammatory bowel disease “ibd”), Cushing's syndrome, cutaneous leukocytoclastic angiitis, dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, eosinophilic pneumonia, epidermolysis bullosa acquisita, erythema nodosum, erythroblastosis fetalis, essential mixed cryoglobulinemia, evan's syndrome, fibrodysplasia ossificans progressiva, fibrosing alveolitis (or idiopathic pulmonary fibrosis), gastritis, gastrointestinal pemphigoid, glomerulonephritis, Goodpasture's syndrome, Graves' disease, Guillain-Barré syndrome (gbs), Hashimoto's encephalopathy, Hashimoto's thyroiditis, Henoch-Schonlein purpura, herpes gestationis aka gestational pemphigoid, Goodpasture's syndrome, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura (autoimmune thrombocytopenic purpura), iga nephropathy, inclusion body myositis, chronic inflammatory demyelinating polyneuropathy, interstitial cystitis, juvenile idiopathic arthritis aka juvenile rheumatoid arthritis, Kawasaki's disease, Lambert-Eaton myasthenic syndrome, leukocytoclastic vasculitis, lichen planus, lichen sclerosus, linear iga disease, lupoid hepatitis aka autoimmune hepatitis, lupus nephritis, Majeed syndrome, Meniere's disease, microscopic polyangiitis, Miller-Fisher syndrome, mixed connective tissue disease, morphea, Mucha-Habermann disease aka pityriasis lichenoides et varioliformis acuta, multiple sclerosis, myasthenia gravis, myositis, narcolepsy, neuromyelitis optica (also Devic's disease), neuromyotonia, occular cicatricial pemphigoid, opsoclonus myoclonus syndrome, ord's thyroiditis, palindromic rheumatism, pandas (pediatric autoimmune neuropsychiatric disorders associated with streptococcus), paraneoplastic cerebellar degeneration, paroxysmal nocturnal hemoglobinuria (pnh), Parry Romberg syndrome, Parsonage-Turner syndrome, pars planitis, pemphigus vulgaris, pernicious anaemia, perivenous encephalomyelitis, Poems syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, rasmussen's encephalitis, raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome, Schnitzler syndrome, scleritis, scleroderma, serum sickness, Sjogren's syndrome, spondyloarthropathy, Still's disease, Stiff person syndrome, subacute bacterial endocarditis, Susac's syndrome, Sweet's syndrome, sydenham chorea, sympathetic ophthalmia, systemic lupus erythematosus, systemic sclerosis, Takayasu's arteritis, temporal arteritis (also known as “giant cell arteritis”), thrombocytopenia, Tolosa-Hunt syndrome, transverse myelitis, ulcerative colitis (one of two types of idiopathic inflammatory bowel disease “ibd”), undifferentiated connective tissue disease, undifferentiated spondyloarthropathy, urticarial vasculitis, vasculitis, vitiligo or Wegener's granulomatosis.

The term “acid sensitive” refers to a binding protein or binding protein conjugate comprising a moiety or plurality of moieties that react under acidic conditions, e.g., within an endosome to release a portion of the binding protein and/or conjugated agent into an endosome. Acid sensitive linkages that can be used to release an active agent in low pH environments, include but are not limited to dimethyl maleic anhydride, cis-aconityl, and hydrazone linkages. Additional examples of acid sensitive compositions can be found in US Publication No. 20110189770.

### II. GENERATION OF BINDING PROTEINS

Binding proteins capable of binding immune cell receptor and/or autoantigen and methods of making the same are provided. The binding protein can be generated using various techniques. Expression vectors, host cell and methods of generating the binding protein are provided and are well known in the art.

A. Criteria for Selecting Parent Monoclonal Antibodies

Certain embodiments is provided comprising selecting parent antibodies with at least one or more properties desired in the bispecific binding protein molecule. In certain embodiments, the desired property is one or more antibody parameters, such as, for example, antigen specificity, affinity to antigen, potency, biological function, epitope recognition, stability, solubility, production efficiency, immunogenicity, pharmacokinetics, bioavailability, tissue cross reactivity, or orthologous antigen binding (e.g., US Patent Publication No. 20090311253).

B. Construction of Binding Protein Molecules

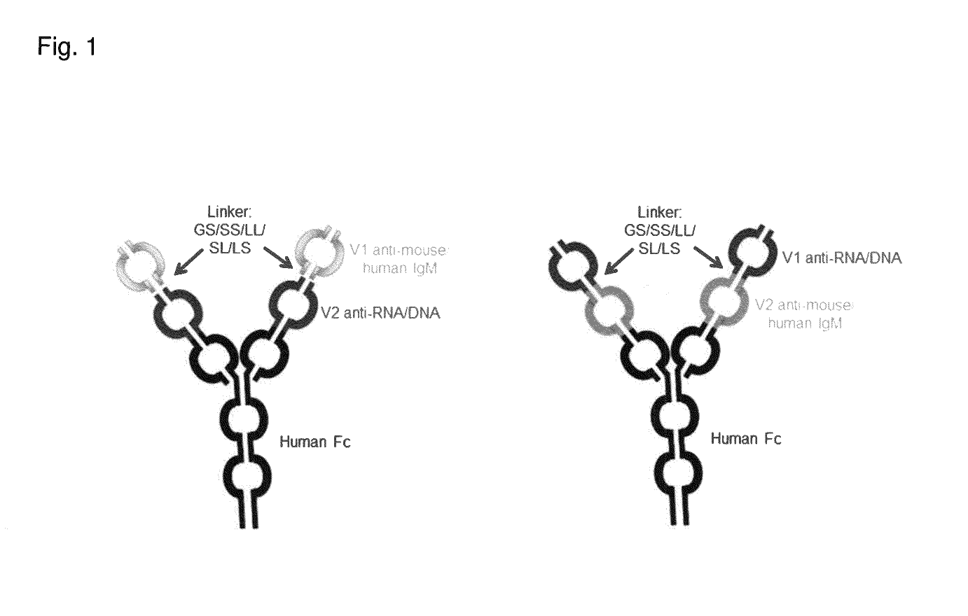

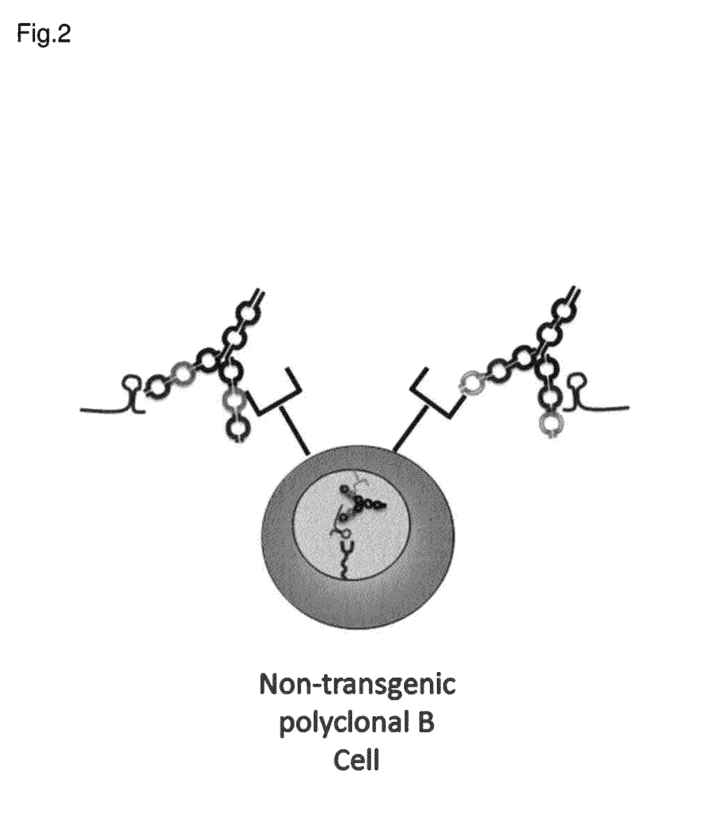

The binding protein may be designed such that two different light chain variable domains (VL) from the two different parent monoclonal antibodies are linked in tandem directly or via a linker by recombinant DNA techniques, followed by the light chain constant domain CL. Similarly, the heavy chain comprises two different heavy chain variable domains (VH) linked in tandem, directly or via a linker, followed by the constant domain CH1 and Fc region (FIG. 1).

The variable domains can be obtained using recombinant DNA techniques from parent antibodies generated by any one of the methods described herein. In certain embodiments, the variable domain is a murine heavy or light chain variable domain. In certain embodiments, the variable domain is a CDR grafted or a humanized variable heavy or light chain domain. In certain embodiments, the variable domain is a human heavy or light chain variable domain.

The linker sequence may be a single amino acid or a polypeptide sequence. In certain embodiments, the choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from the C-terminus of a V domain and 4-6 residues from the N-terminus of a CL/CH1 domain. The N-terminal residues of CL or CH1 domains, particularly the first 5-6 amino acid residues, can adopt a loop conformation without strong secondary structures, and therefore can act as flexible linkers between the two variable domains. The N-terminal residues of CL or CH1 domains are natural extension of the variable domains, as they are part of the Ig sequences, and therefore their use may minimize immunogenicity.

In certain embodiments, the heavy chain, light chain, two chain, or four chain embodiments include at least one linker comprising the amino acid sequence AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); GGGGSGGGGS (SEQ ID NO: 29); GGSGGGGSG (SEQ ID NO: 30); or G/S based sequences (e.g., G4S and G4S repeats; SEQ ID NO: 31). In certain embodiments, X2 is an Fc region. In certain embodiments, X2 is a variant Fc region.

Other linker sequences may include any sequence of any length of a CL/CH1 domain but not all residues of a CL/CH1 domain; for example the first 5-12 amino acid residues of a CL/CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR); hinge region-derived sequences; and other natural sequences from other proteins.

In certain embodiments, a constant domain is linked to the two linked variable domains using recombinant DNA techniques. In certain embodiments, a sequence comprising linked heavy chain variable domains is linked to a heavy chain constant domain and a sequence comprising linked light chain variable domains is linked to a light chain constant domain. In certain embodiments, the constant domains are human heavy chain constant domains and human light chain constant domains respectively. In certain embodiments, the bispecific binding protein heavy chain is further linked to an Fc region. The Fc region may be a native sequence Fc region or a variant Fc region. In certain embodiments, the Fc region is a human Fc region. In certain embodiments, the Fc region includes Fc region from IgG1, IgG2, IgG3, IgG4, IgA, IgM, IgE, or IgD.

In certain embodiments, two heavy chain bispecific polypeptides and two light chain bispecific polypeptides are combined to form a bispecific binding protein of the invention. Table 1 lists amino acid sequences of VH and VL regions of exemplary parent antibodies useful for making the bispecific binding proteins disclosed herein. In certain embodiments, a bispecific binding protein comprising at least two of the VH and/or VL regions listed in Table 1, in any orientation, is provided. The VH and VL domain sequences comprise complementarity determining regions (CDRs) (bold) and framework sequences that are either known in the art or readily discernible using methods known in the art. In certain embodiments, one or more of these CDRs and/or framework sequences are replaced, without loss of function, by other CDRs and/or framework sequences from binding proteins that are known in the art to bind to the same antigen.

TABLE 1
List Of Amino Acid Sequences Of VH And VL Regions Of Antibodies
For Generating Binding Proteins

| | ABT | | Sequence |
|---|---|---|---|
| SEQ ID No. | Unique ID | Protein | 123456789012345678901234567890 |
| 32 | AB529VH | DNA (PA4) | EVQLVESGGGLVKPGGSLKLSCVASGFTFS |
| | | | TYAMSWVRQTPTKRLEWVATISRGGVSTYY |
| | | | PDTVKGRFTISRDNAKNTLYLQMSSLRSED |
| | | | TAMYYCARPPTIVTTWFAYWGQGTLVTVSS |
| CDR1: Amino | | | TYAMS |
| Acids 31-35 of | | | |
| SEQ ID NO. 32 | | | |
| CDR 2: Amino | | | TISRGGVSTYYPDTVKG |
| Acids50-66 of | | | |
| SEQ ID NO. 32 | | | |
| CDR3: Amino | | | PPTIVTTWFAY |
| Acids99-109 of | | | |
| SEQ ID NO. 32 | | | |
| 33 | AB529VL | DNA (PA4) | DVVMTQTPLSLPVSLGDQASISCRSSQSLV |
| | | | HSNGNTYLHWYLQKPGQSPKLLIYKVSNRF |
| | | | SGVPDRFSGSGSGTDFTLKISRVEAEDLGA |
| | | | YFCSQSTHVPYTFGGPTRLEIKR |
| CDR1: Amino | | | RSSQSLVHSNGNTYLH |
| Acids24-39 of | | | |
| SEQ ID NO. 33 | | | |
| CDR 2: Amino | | | KVSNRFS |
| Acids55-61 of | | | |
| SEQ ID NO. 33 | | | |
| CDR3: Amino | | | SQSTHVPYT |
| Acids94-102 of | | | |
| SEQ ID NO. 33 | | | |
| 34 | AB531VH | mIgM | QVQLKESGPGLVKPSLTLSLTCTVSGFSLN |
| | | (B7.6) | GYGVIWVRQPPGKGLEWMGVIWGNGNTNYN |
| | | | STLKSRLSISRDTSKSQVFLKMNNLQTEDT |
| | | | AMYFCARSENYYSSPGYFAYWGQGTLVTVS |
| | | | S |
| CDR1: Amino | | | GYGVI |
| Acids31-35 of | | | |
| SEQ ID NO. 34 | | | |
| CDR 2: Amino | | | VIWGNGNTNYNSTLKS |
| Acids50-65 of | | | |
| SEQ ID NO. 34 | | | |
| CDR3: Amino | | | SENYYSSPGYFAY |
| Acids98-110 of | | | |
| SEQ ID NO. 34 | | | |
| 35 | AB531VL | mIgM | NTVMTQSPTSMFISVGDRVTMNCKASQNVG |
| | | (B7.6) | SDVDWYQQKTGQSPKLLISGTSNRYTGVPD |
| | | | RFTGSGSGTDFTLTISNMQAEDLAVYYCLQ |
| | | | YNYNPTFGAGTKLELKR |
| CDR1: Amino | | | KASQNVGSDVD |
| Acids24-34 of | | | |
| SEQ ID NO. 35 | | | |
| CDR 2: Amino | | | GTSNRYT |
| Acids50-56 of | | | |
| SEQ ID NO. 35 | | | |
| CDR3: Amino | | | LQYNYNPT |
| Acids89-96 of | | | |
| SEQ ID NO. 35 | | | |
| 36 | AB530VH | RNA (BWR4) | EVQLQQSGPEPAKPGASVKMSCKASGYTFT |
| | | | SSVIHWVKQKPGQGLEWIGYINPYNDDTKY |
| | | | NEKFKGKATLTSDKSSSTAYMELSSLTSED |
| | | | SGVYYCARRLRLFAYWGQGTLVTVSA |
| CDR1: Amino | | | SSVIH |
| Acids of | | | |
| SEQ ID NO. 36 | | | |
| CDR 2: Amino | | | YINPYNDDTKYNEKFKG |
| Acids of | | | |
| SEQ ID NO. 36 | | | |
| CDR3: Amino | | | RLRLFAY |
| Acids of | | | |
| SEQ ID NO. 3 | | | |
| 37 | AB530VL | RNA | EIVLTQSPALMAASPGEKVTITCSVSSSIS |
| | | (BWR4) | SSNLHWYQQKSESSPKPWIYGTSNLASGVP |
| | | | VRFSGSGSGTSYSLTISSMEAEDAATYYCQ |
| | | | QWSGYPLTFGSGTKLEIKR |
| CDR1: Amino | | | SVSSSISSSNLH |
| Acids of | | | |
| SEQ ID NO. 37 | | | |
| CDR 2: Amino | | | GTSNLAS |
| Acids of | | | |
| SEQ ID NO. 37 | | | |
| CDR3: Amino | | | QQWSGYPLT |
| Acids of | | | |
| SEQ ID NO. 37 | | | |
| 38 | AB532VH | hIgM | QVQLQQPGAEFVKPGAPVKLSCKASGYPFT |
| | | (HB-57.3) | TYWVNWMKQRPGRGLEWIGRIDPYDSETLY |
| | | | NQKFKDKATLIVDKSSSTAYIQLSSLTSED |
| | | | SAVYYCARETYDYPFAYWGQGTLVTVSS |
| CDR1: Amino | | | TYWVN |
| Acids of | | | |
| SEQ ID NO. 38 | | | |
| CDR 2: Amino | | | RIDPYDSETLYNQKFKD |
| Acids of | | | |
| SEQ ID NO. 38 | | | |
| CDR3: Amino | | | ETYDYPFAY |
| Acids of | | | |
| SEQ ID NO. 38 | | | |
| 39 | AB532VL | hIgM | DIVMTQSPSSLAMSVGQKVTMSCKSSQSLL |
| | | (HB-57.3) | NSSNQKNYLAWYQQKPGQSPELLVYFASTR |
| | | | ESGVPDRFIGSGSGTDFTLTISSVQAEDLA |
| | | | DYFCQQHYSTPFTFGSETKLEIKR |
| CDR1: Amino | | | KSSQSLLNSSNQKNYLA |
| Acids of | | | |
| SEQ ID NO. 39 | | | |
| CDR 2: Amino | | | FASTRES |
| Acids of | | | |
| SEQ ID NO. 39 | | | |
| CDR3: Amino | | | QQHYSTPFT |
| Acids of | | | |
| SEQ ID NO. 39 | | | |

Detailed description of bispecific binding proteins capable of binding specific targets, and methods of making the same, is provided in the Examples section below.

C. Production of Binding Proteins

The bispecific binding proteins may be produced by any of a number of techniques known in the art. For example, expression from host cells, wherein expression vector(s) encoding the bispecific binding protein heavy and bispecific binding protein light chains is (are) transfected into a host cell by standard techniques. Although it is possible to express the bispecific binding protein in either prokaryotic or eukaryotic host cells, bispecific binding proteins are expressed in eukaryotic cells, for example, mammalian host cells, because such eukaryotic cells (and in particular mammalian cells) are more likely than prokaryotic cells to assemble and secrete a properly folded and immunologically active bispecific binding proteins.

In an exemplary system for recombinant expression of bispecific binding proteins, a recombinant expression vector encoding both the bispecific binding protein heavy chain and the bispecific binding protein light chain is introduced into dhfr− CHO cells by calcium phosphate-mediated transfection. Within the recombinant expression vector, the bispecific binding protein heavy and light chain sequences are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the bispecific binding protein heavy and light chains and intact bispecific binding protein is recovered from the culture medium. Standard molecular biology techniques are used to prepare the recombinant expression vector, transfect the host cells, select for transformants, culture the host cells and recover the bispecific binding protein from the culture medium. A method of synthesizing a bispecific binding protein by culturing a host cell in a suitable culture medium until a bispecific binding protein is synthesized is also provided. The method can further comprise isolating the bispecific binding protein from the culture medium.

An important feature of bispecific binding protein is that it can be produced and purified in a similar way as a conventional antibody. The production of bispecific binding proteins results in a homogeneous, single major product with desired dual-specific activity, without the need for sequence modification of the constant region or chemical modifications. Other previously described methods to generate “bi-specific”, “multi-specific”, and “multi-specific multivalent” full length binding proteins can lead to the intracellular or secreted production of a mixture of assembled inactive, mono-specific, multi-specific, multivalent, full length binding proteins, and multivalent full length binding proteins with a combination of different binding sites.

Surprisingly, the design of the “dual-specific multivalent full length binding proteins” provided herein leads to a dual variable domain light chain and a dual variable domain heavy chain that assemble primarily to the desired “dual-specific multivalent full length binding proteins”.

At least 50%, at least 75% and at least 90% of the assembled, and expressed dual variable domain immunoglobulin molecules are the desired dual-specific tetravalent protein, and therefore possess enhanced commercial utility. Thus, a method to express a dual variable domain light chain and a dual variable domain heavy chain in a single cell leading to a single primary product of a “dual-specific tetravalent full length binding protein” is provided.

Methods of expressing a dual variable domain light chain and a dual variable domain heavy chain in a single cell leading to a “primary product” of a “dual-specific tetravalent full length binding protein”, where the “primary product” is more than 50%, such as more than 75% and more than 90%, of all assembled protein, comprising a dual variable domain light chain and a dual variable domain heavy chain are provided.

### III. USES OF BISPECIFIC BINDING PROTEINS

Given their ability to bind to two or more antigens the binding proteins provided herein can be used to detect the antigens (e.g., in a biological sample, such as serum or plasma), using a conventional immunoassay, such as an enzyme linked immunosorbent assays (ELISA), a radioimmunoassay (RIA), or tissue immunohistochemistry. The bispecific binding protein is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol and examples of suitable radioactive materials include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In certain embodiments, the bispecific binding proteins provided herein are capable of neutralizing the activity of their antigen targets both in vitro and in vivo. Accordingly, such bispecific binding proteins can be used to inhibit antigen activity, e.g., in a cell culture containing the antigens, in human subjects or in other mammalian subjects having the antigens with which a bispecific binding protein provided herein cross-reacts. In certain embodiments, a method for reducing antigen activity in a subject suffering from a disease or disorder in which the antigen activity is detrimental is provided. A bispecific binding protein provided herein can be administered to a human subject for therapeutic purposes.

The term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins provided herein include those disorders discussed below and in the section pertaining to pharmaceutical compositions comprising the binding proteins. In certain embodiments, the antigen comprises DNA and/or RNA.

Bispecific binding proteins are useful as therapeutic agents to simultaneously block two different targets to enhance efficacy/safety and/or increase patient coverage.

Additionally, bispecific binding proteins provided herein can be employed for tissue-specific delivery (target a tissue marker and a disease mediator for enhanced local PK thus higher efficacy and/or lower toxicity), including intracellular delivery (targeting an internalizing receptor and an intracellular molecule), delivering to inside brain (targeting transferrin receptor and a CNS disease mediator for crossing the blood-brain barrier). Bispecific binding proteins can also serve as a carrier protein to deliver an antigen to a specific location via binding to a non-neutralizing epitope of that antigen and also to increase the half-life of the antigen. Furthermore, bispecific binding protein can be designed to either be physically linked to medical devices implanted into patients or target these medical devices (Burke et al. (2006) Adv. Drug Deliv. Rev. 58(3):437-446; Hildebrand et al. (2006) Surface and Coatings Technol. 200(22-23):6318-6324; Drug/device combinations for local drug therapies and infection prophylaxis, Wu (2006) Biomater. 27(11):2450-2467; Mediation of the cytokine network in the implantation of orthopedic devices (Marques (2005) Biodegrad. Sys. Tissue Engineer. Regen. Med. vol:377-397). Briefly, directing appropriate types of cell to the site of medical implant may promote healing and restoring normal tissue function. Alternatively, inhibition of mediators (including but not limited to cytokines), released upon device implantation by a bispecific binding protein coupled to or target to a device is also provided. In certain embodiments, the bispecific binding protein triggers a TLR, such as TLR7 and TLR9, for example, in a B cell.

A. Use of Binding Proteins in Various Diseases

Binding proteins provided herein are useful as therapeutic molecules to treat various diseases, e.g., wherein the targets that are recognized by the binding proteins are detrimental. Such binding proteins may bind one or more targets involved in a specific disease Inhibition of an immune cell receptor and/or autoantigen has also been shown to enhance anti-viral vaccines in animal models and may be beneficial in the treatment of HIV and other infectious diseases, for example, the human rhinovirus, other enteroviruses, coronavirus, herpes viruses, influenza virus, parainfluenza virus, respiratory syncytial virus or adenovirus.

Without limiting the disclosure, further information on certain disease conditions is provided.

1. Human Autoimmune and Inflammatory Response

Immune cell receptors and/or autoantigens have been implicated in general autoimmune and inflammatory responses, including, for example, asthma, allergies, allergic lung disease, allergic rhinitis, atopic dermatitis, chronic obstructive pulmonary disease (COPD), fibrosis, cystic fibrosis (CF), fibrotic lung disease, idiopathic pulmonary fibrosis, liver fibrosis, lupus, hepatitis B-related liver diseases and fibrosis, sepsis, systemic lupus erythematosus (SLE), glomerulonephritis, inflammatory skin diseases, psoriasis, diabetes, insulin dependent diabetes mellitus, inflammatory bowel disease (IBD), ulcerative colitis (UC), Crohn's disease (CD), rheumatoid arthritis (RA), osteoarthritis (OA), multiple sclerosis (MS), graft-versus-host disease (GVHD), transplant rejection, ischemic heart disease (IHD), celiac disease, contact hypersensitivity, alcoholic liver disease, Behcet's disease, atherosclerotic vascular disease, occular surface inflammatory diseases, or Lyme disease.

The bispecific binding proteins provided herein can be used to treat neurological disorders. In certain embodiments, the bispecific binding proteins provided herein or antigen binding portions thereof, are used to treat neurodegenerative diseases, and conditions involving neuronal regeneration and spinal cord injury.

2. Rheumatoid Arthritis

Rheumatoid arthritis (RA), a systemic disease, is characterized by a chronic inflammatory reaction in the synovium of joints and is associated with degeneration of cartilage and erosion of juxta-articular bone. Many pro-inflammatory cytokines, chemokines, and growth factors are expressed in diseased joints. Whether a binding protein molecule will be useful for the treatment of rheumatoid arthritis can be assessed using pre-clinical animal RA models such as the collagen-induced arthritis mouse model. Other useful models are also well known in the art (Brand (2005) Comp. Med. 55(2):114-22). Based on the cross-reactivity of the parental antibodies for human and mouse orthologues (e.g., reactivity for human and mouse TNF, human and mouse IL-15, etc.) validation studies in the mouse CIA model may be conducted with “matched surrogate antibody” derived binding protein molecules; briefly, a binding protein based on two (or more) mouse target specific antibodies may be matched to the extent possible to the characteristics of the parental human or humanized antibodies used for human binding protein construction (e.g., similar affinity, similar neutralization potency, similar half-life, etc.).

3. Systemic Lupus Erythematosus (SLE)

Systemic lupus erythematosus (SLE) is a complicated autoimmune disease diagnosed on presentation of a variable subset of a wide array of clinical symptoms. The feature common to all SLE patients, however, is the presence of an autoimmune response to nuclear antigens. Although self-reactive B cells produce the autoantibodies essential to the diagnosis of disease, B cells have proven to be active participants in the development of disease irrespective of autoantibody production. A central question surrounding the pathogenesis of the disease is whether intrinsic defects in SLE B cells play a role in triggering the immunological events that result in the onset of clinical disease. Although other immune cells play a role in SLE, B cells from SLE patients display signaling defects that appear to underlie pathogenesis and explain the characteristic hyperactivity of B cells in active disease that ultimately leads to a breakdown of B cell tolerance and the subsequent pathogenesis of SLE.

An immunopathogenic hallmark of SLE is the polyclonal B cell activation, which leads to hyperglobulinemia, autoantibody production and immune complex formation. The Toll-like receptors (TLRs), which play a key role in innate responses to infections, are also involved in acute and chronic inflammatory processes induced by endogenous ligands.

In particular, the endosomally localized TLR7 and TLR9 are activated by autoimmune complexes containing self RNA and DNA in B lymphocytes and dendritic cells, respectively. These endogenous TLR ligands act as autoadjuvants providing a stimulatory signal together with the autoantigen and thus contribute to break peripheral tolerance against self antigens in SLE, for example. In vivo studies in SLE mouse models demonstrate an essential role for TLR7 in the generation of RNA-containing antinuclear antibodies and deposition of pathogenic immune complexes in the kidney. DNA-reactive TLR9, however, appears to have immunostimulatory as well as regulatory functions in SLE mouse models. Type I Interferon, which is produced by plasmacytoid dendritic cells in response to autoimmune complexes containing RNA and DNA recognized by TLR7 and TLR9 acts as a potent amplifier of the autoimmune response. TLR-independent recognition of self nucleic acids by cytosolic RNA and DNA sensors may also play a role in the generation of autoimmune responses (Krug (2008) Handbook Exp. Pharmacol. (183):129-51).

Significant increased levels of IL-17 have been detected in patients with systemic lupus erythematosus (Morimoto et al. (2001) Autoimmun 34(1):19-25; Wong et al. (2008) Clin. Immunol. 127(3):385-93). IL-17 represents an important cytokine in the pathogenesis of SLE. Increased IL-17 production has been shown in patients with SLE as well as in animals with lupus-like diseases. Animal models have demonstrated that blockade of IL-17 decreases lupus manifestations (for a review see Nalbandian et al. (2009) 157(2):209-215). Based on the cross-reactivity of the parental antibodies for human and mouse othologues (e.g., reactivity for human and mouse CD20, human and mouse interferon alpha, etc.) validation studies in a mouse lupus model may be conducted with “matched surrogate antibody” derived binding protein molecules. Briefly, a binding protein based two (or more) mouse target specific antibodies may be matched to the extent possible to the characteristics of the parental human or humanized antibodies used for human binding protein construction (e.g., similar affinity, similar neutralization potency, similar half-life, etc.).

### IV. PHARMACEUTICAL COMPOSITIONS

Pharmaceutical compositions comprising one or more binding proteins, either alone or in combination with prophylactic agents, therapeutic agents, and/or pharmaceutically acceptable carriers are provided. The pharmaceutical compositions comprising binding proteins provided herein are for use in, but not limited to, diagnosing, detecting, prognosing, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art (US Patent Publication No. 20090311253).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (US Patent Publication No. 20090311253).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-20 mg/kg, for example, 1-10 mg/kg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### V. COMBINATION THERAPY

A binding protein provided herein also can also be administered with one or more additional therapeutic agents useful in the treatment of various diseases, the additional agent being selected by the skilled artisan for its intended purpose. For example, the additional agent can be a therapeutic agent art-recognized as being useful to treat the disease or condition being treated by the antibody provided herein. The combination can also include more than one additional agent, e.g., two or three additional agents.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

Combinations to treat autoimmune and inflammatory diseases are non-steroidal anti-inflammatory drug(s) also referred to as NSAIDS which include drugs like ibuprofen. Other combinations are corticosteroids including prednisolone; the well known side-effects of steroid use can be reduced or even eliminated by tapering the steroid dose required when treating patients in combination with the binding proteins provided herein. Non-limiting examples of therapeutic agents for rheumatoid arthritis with which an antibody provided herein, or antibody binding portion thereof, can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Binding proteins provided herein, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Combinations of therapeutic agents may interfere at different points in the autoimmune and subsequent inflammatory cascade. Examples include a binding protein disclosed herein and a TNF antagonist like a chimeric, humanized or human TNF antibody, Adalimumab, (PCT Publication No. WO 97/29131), CA2 (Remicade™), CDP 571, a soluble p55 or p75 TNF receptor, or derivative thereof (p75TNFR1gG (Enbrel™) or p55TNFR1gG (Lenercept)), a TNFα converting enzyme (TACE) inhibitor; or an IL-1 inhibitor (an Interleukin-1-converting enzyme inhibitor, IL-1RA, etc.). Other combinations include a binding protein disclosed herein and Interleukin 11. Yet another combination include key players of the autoimmune response which may act parallel to, dependent on or in concert with IL-12 function; especially relevant are IL-18 antagonists including an IL-18 antibody, a soluble IL-18 receptor, or an IL-18 binding protein. It has been shown that IL-12 and IL-18 have overlapping but distinct functions and a combination of antagonists to both may be most effective. Yet another combination is a binding protein disclosed herein and a non-depleting anti-CD4 inhibitor. Yet other combinations include a binding protein disclosed herein and an antagonist of the co-stimulatory pathway CD80 (B7.1) or CD86 (B7.2) including an antibody, a soluble receptor, or an antagonistic ligand.

The binding proteins provided herein may also be combined with an agent, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, a corticosteroid (oral, inhaled and local injection), a beta-2 adrenoreceptor agonist (salbutamol, terbutaline, salmeteral), a xanthine (theophylline, aminophylline), cromoglycate, nedocromil, ketotifen, ipratropium, oxitropium, cyclosporin, FK506, rapamycin, mycophenolate mofetil, leflunomide, an NSAID, for example, ibuprofen, a corticosteroid such as prednisolone, a phosphodiesterase inhibitor, an adensosine agonist, an antithrombotic agent, a complement inhibitor, an adrenergic agent, an agent which interferes with signalling by proinflammatory cytokines such as TNF-α or IL-1 (e.g., IRAK, NIK, IKK, p38 or a MAP kinase inhibitor), an IL-1β converting enzyme inhibitor, a TNFα converting enzyme (TACE) inhibitor, a T-cell signalling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivative thereof (e.g., a soluble p55 or p75 TNF receptor or the derivative p75TNFRIgG (Enbrel™) or p55TNFRIgG (Lenercept), sIL-1RI, sIL-1RII, sIL-6R), an antiinflammatory cytokine (e.g., IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, or Mesopram. Combinations include methotrexate or leflunomide and in moderate or severe rheumatoid arthritis cases, cyclosporine.

In certain embodiments, the binding protein or antigen binding portion thereof, is administered in combination with one of the following agents for the treatment of rheumatoid arthritis: a small molecule inhibitor of KDR, a small molecule inhibitor of Tie-2; methotrexate; prednisone; celecoxib; folic acid; hydroxychloroquine sulfate; rofecoxib; etanercept; infliximab; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1 TRAP; MRA; CTLA4-IG; IL-18 BP; IL-12/23; anti-IL 18; anti-IL 15; BIRB-796; SCIO-469; VX-702; AMG-548; VX-740; Roflumilast; IC-485; CDC-801; or mesopram.

Non-limiting examples of therapeutic agents for multiple sclerosis with which binding proteins provided herein can be combined include the following: a corticosteroid; prednisolone; methylprednisolone; azathioprine; cyclophosphamide; cyclosporine; methotrexate; 4-aminopyridine; tizanidine; interferon-β1a (AVONEX; Biogen); interferon-β1b (BETASERON; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE; Teva Pharmaceutical Industries, Inc.); hyperbaric oxygen; intravenous immunoglobulin; clabribine; an antibody to or antagonist of other human cytokines or growth factors and their receptors, for example, TNF, LT, IL-1, IL-2, IL-6, IL-7, IL-8, IL-23, IL-15, IL-16, IL-18, EMAP-II, GM-CSF, FGF, or PDGF. Binding proteins provided herein can be combined with an antibody to a cell surface molecule such as CD2, CD3, CD4, CD8, CD19, CD20, CD25, CD28, CD30, CD40, CD45, CD69, CD80, CD86, CD90 or their ligands. Binding proteins provided herein, may also be combined with an agent, such as methotrexate, cyclosporine, FK506, rapamycin, mycophenolate mofetil, leflunomide, an NSAID, for example, ibuprofen, a corticosteroid such as prednisolone, a phosphodiesterase inhibitor, an adensosine agonist, an antithrombotic agent, a complement inhibitor, an adrenergic agent, an agent which interferes with signalling by a proinflammatory cytokine such as TNFα or IL-1 (e.g., IRAK, NIK, IKK, p38 or a MAP kinase inhibitor), an IL-1β converting enzyme inhibitor, a TACE inhibitor, a T-cell signaling inhibitor such as a kinase inhibitor, a metalloproteinase inhibitor, sulfasalazine, azathioprine, a 6-mercaptopurine, an angiotensin converting enzyme inhibitor, a soluble cytokine receptor or derivatives thereof (e.g., a soluble p55 or p75 TNF receptor, sIL-1RI, sIL-1RII, sIL-6R), an antiinflammatory cytokine (e.g., IL-4, IL-10, IL-13 or TGFβ) or a bcl-2 inhibitor.

Examples of therapeutic agents for SLE (Lupus) in which binding proteins provided herein can be combined include the following: NSAIDS, for example, diclofenac, naproxen, ibuprofen, piroxicam, indomethacin; COX2 inhibitors, for example, Celecoxib, rofecoxib, valdecoxib; anti-malarials, for example, hydroxychloroquine; Steroids, for example, prednisone, prednisolone, budenoside, dexamethasone; Cytotoxics, for example, azathioprine, cyclophosphamide, mycophenolate mofetil, methotrexate; inhibitors of PDE4 or purine synthesis inhibitor, for example Cellcept. Binding proteins provided herein may also be combined with agents such as sulfasalazine, 5-aminosalicylic acid, olsalazine, Imuran and agents which interfere with synthesis, production or action of proinflammatory cytokines such as IL-1, for example, caspase inhibitors like IL-1β converting enzyme inhibitors and IL-1ra. Binding proteins provided herein may also be used with T cell signaling inhibitors, for example, tyrosine kinase inhibitors; or molecules that target T cell activation molecules, for example, CTLA-4-IgG or anti-B7 family antibodies, anti-PD-1 family antibodies. Binding proteins provided herein, can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. Antibodies provided herein or antigen binding portion thereof may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, Adalimumab (PCT Publication No. WO 97/29131; HUMIRA), CA2 (REMICADE), CDP 571, TNFR-Ig constructs, (p75TNFRIgG (ENBREL) and p55TNFRIgG (LENERCEPT)) and bcl-2 inhibitors, because bcl-2 overexpression in transgenic mice has been demonstrated to cause a lupus like phenotype (Gonzales et al. (2007) J. Immunol. 178(5):2778-86). The pharmaceutical compositions provided herein may include a “therapeutically effective amount” or a “prophylactically effective amount” of a binding protein provided herein. A “therapeutically effective amount” refers to an amount effective, at dosages and for periods of time necessary, to achieve the desired therapeutic result. A therapeutically effective amount of the binding protein may be determined by a person skilled in the art and may vary according to factors such as the disease state, age, sex, and weight of the individual, and the ability of the binding protein to elicit a desired response in the individual. A therapeutically effective amount is also one in which any toxic or detrimental effects of the antibody, or antibody binding portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### VI. DIAGNOSTICS AND PROGNOSTICS

The disclosure herein also provides diagnostic/prognostic applications including, but not limited to, diagnostic assay methods, diagnostic kits containing one or more binding proteins, and adaptation of the methods and kits for use in automated and/or semi-automated systems. The methods, kits, and adaptations provided may be employed in the detection, monitoring, and/or treatment of a disease or disorder in an individual. This is further elucidated below.

A. Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure (US Patent Publication No. 20090311253).

B. Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art (US Patent Publication No. 20090311253).

C. Adaptation of Kit and Method

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other platforms available from Abbott Laboratories include, but are not limited to, AxSYM®, IMx® (e.g., U.S. Pat. No. 5,294,404), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays Immunosensors and their methods of manufacture and operation in single-use test devices are described, for example in, U.S. Pat. Nos. 5,063,081; 7,419,821, and 7,682,833; and US Publication Nos. 20040018577; 20060160164; and 20090311253.

It will be readily apparent to those skilled in the art that other suitable modifications and adaptations of the compositions and methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### Generation and Characterization of Anti-Mouse IgM and Anti-DNA Dual Variable Domain Immunoglobulin (DVD-Ig™) Protein

Four-chain dual variable domain immunoglobulin (DVD-Ig™) proteins were generated by synthesizing polynucleotide fragments encoding immunoglobulin variable heavy chain and variable light chain sequences and cloning the fragments into a pCDNA 3.3 vector (Life Technologies). The DVD-Ig™ constructs were cloned into and expressed in human embryonic kidney 293 cells and purified according to art known methods. VH and VL chain amino acid sequences for the DVD-Ig™ proteins are provided in Table 1. The SEQ ID NOs listed in the leftmost column of Table 2 refer to the sequences for the full variable domain of the heavy and light chains of the DVD-Ig™ proteins in that row of the Table. Each row in the rightmost column of Table 2 provides three SEQ ID NOs. The first number refers to the SEQ ID NO of the outer variable domain sequence, the second number refers to the SEQ ID NO of the linker, and the third number refers to the SEQ ID NO of the inner variable domain sequence, that together are found within the full DVD-Ig™ variable domain sequences (i.e., each of the heavy and light variable domain of the full DVD-Ig™ protein comprising VD1-X1-VD2).

TABLE 2
DVD-Ig ™ Binding Proteins That Bind Mouse IgM and DNA

| | DVD- | Outer | | Inner | |
|---|---|---|---|---|---|
| | Ig | Variable | | Variable | SEQ ID |
| SEQ | Variable | Domain | | Domain | NO VD1- |
| ID | Domain | Name | | Name | X1-VD2 |
| NO | Name | (VD1) | Linker | (VD2) | Formula |
| 40 | DVD3746 | hIgM (HB- | GS | RNA (BWR4) | H: 38-29-36 |
| 41 | | 57.3) | | | L: 39-30-37 |
| 42 | DVD3747 | hIgM (HB- | SS | RNA (BWR4) | H: 38-21-36 |
| 43 | | 57.3) | | | L: 39-13-37 |
| 44 | DVD- | hIgM (HB- | SL | RNA (BWR4) | H: 38-22-36 |
| 45 | 3749 | 57.3) | | | L: 39-14-37 |
| 46 | DVD- | hIgM (HB- | LS | RNA (BWR4) | H: 38-22-36 |
| 47 | 3750 | 57.3) | | | L: 39-13-37 |
| 48 | DVD3751 | DNA(PA4) | GS | mIgM | H: 32-29-34 |
| 49 | | | | (B7.6) | L: 33-30-35 |
| 50 | DVD3752 | DNA(PA4) | SS | mIgM | H: 32-21-34 |
| 51 | | | | (B7.6) | L: 33-13-35 |
| 52 | DVD3753 | DNA(PA4) | LL | mIgM | H: 32-22-34 |
| 53 | | | | (B7.6) | L: 33-14-35 |
| 54 | DVD3754 | DNA(PA4) | SL | mIgM | H: 32-21-34 |
| 55 | | | | (B7.6) | L: 33-14-35 |
| 56 | DVD3755 | DNA(PA4) | LS | mIgM | H: 32-22-34 |
| 57 | | | | (B7.6) | L: 33-13-35 |
| 58 | DVD3756 | mIgM | GS | DNA(PA4) | H: 34-29-32 |
| 59 | | (B7.6) | | | L: 35-30-33 |
| 60 | DVD3757 | mIgM | SS | DNA(PA4) | H: 34-21-32 |
| 61 | | (B7.6) | | | L: 35-13-33 |
| 62 | DVD3758 | mIgM | LL | DNA(PA4) | H: 34-22-32 |
| 63 | | (B7.6) | | | L: 35-14-33 |
| 64 | DVD3759 | mIgM | SL | DNA(PA4) | H: 34-21-32 |
| 65 | | (B7.6) | | | L: 35-14-33 |
| 66 | DVD3760 | mIgM | LS | DNA(PA4) | H: 34-22-32 |
| 67 | | (B7.6) | | | L: 35-13-33 |
| 68 | | DNA (PA4) | GS | hIgM (HB- | H: 32-29-38 |
| 69 | DVD3761 | | | 57.3) | L: 33-30-39 |
| 70 | | DNA (PA4) | SS | hIgM (HB- | H: 32-21-38 |
| 71 | DVD3762 | | | 57.3) | L: 33-13-39 |
| 72 | | DNA (PA4) | SL | hIgM (HB- | H: 32-21-38 |
| 73 | DVD3764 | | | 57.3) | L: 33-14-39 |
| 74 | | DNA (PA4) | LS | hIgM (HB- | H: 32-22-38 |
| 75 | DVD3765 | | | 57.3) | L: 33-13-39 |
| 76 | | hIgM (HB- | GS | DNA (PA4) | H: 38-29-32 |
| 77 | DVD3766 | 57.3) | | | L: 39-30-33 |
| 78 | | hIgM (HB- | SS | DNA (PA4) | H: 38-21-32 |
| 79 | DVD3767 | 57.3) | | | L: 39-13-33 |
| 80 | | hIgM (HB- | SL | DNA (PA4) | H: 38-21-32 |
| 81 | DVD3769 | 57.3) | | | L: 39-14-33 |
| 82 | | hIgM (HB- | LS | DNA (PA4) | H: 38-22-32 |
| 83 | DVD3770 | 57.3) | | | L: 39-13-33 |

The DVD-Ig binding proteins listed above comprise a human light chain kappa constant region (SEQ ID NO: 84) and the wild type hIgG1 constant region (SEQ ID NO: 85). The constant domain sequences and alternatives are shown below in Table 3.

TABLE 3
Human IgG Heavy and Light Chain Constant Domains

| | | Sequence |
|---|---|---|
| Protein | SEQ ID NO | 1234567890123456789012345678901234567890 |
| Wild type | 85 | ASTKGPSVFPLAPSSKSTSGGTAALGCLVKDYFPEPVTVS |
| hIgG1 | | WNSGALTSGVHTFPAVLQSSGLYSLSSVVTVPSSSLGTQT |
| constant region | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| Mutant hIgG1 | 86 | ASTKGPSVFPLAPSSKSTSGGTAALGCLVKDYFPEPVTVS |
| constant region | | WNSGALTSGVHTFPAVLQSSGLYSLSSVVTVPSSSLGTQT |
| (IgG1, z, | | YICNVNHKPSNTKVDKKVEPKSCDKTHTCPPCPAPEAAGG |
| non-a mut | | PSVFLFPPKPKDTLMISRTPEVTCVVVDVSHEDPEVKFNW |
| (234, 235)) | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| Ig kappa | 84 | TVAAPSVFIFPPSDEQLKSGTASVVCLLNNFYPREAKVQW |
| constant region | | KVDNALQSGNSQESVTEQDSKDSTYSLSSTLTLSKADYEK |
| | | HKVYACEVTHQGLSSPVTKSFNRGEC |
| Ig Lambda | 87 | QPKAAPSVTLFPPSSEELQANKATLVCLISDFYPGAVTVA |
| constant region | | WKADSSPVKAGVETTTPSKQSNNKYAASSYLSLTPEQWKS |
| | | HRSYSCQVTHEGSTVEKTVAPTECS |

### Example 2

### Assays Used to Determine the Functional Activity of Parent Antibodies and DVD-Ig™ Proteins

### Example 2.1

### Mice

The AM14 B cell receptor (BCR) transgenic mouse has been described previously (Sweet et al. (2010) Autoimmun 43(8): 607-18). Briefly, AM14 is a BCR comprising the AM14 heavy chain and the Vk8 light chain that recognizes murine IgG2a (mIgG2a) of the “a” allotype. Between 95-98% of B cells in a mouse positive for the AM14 heavy chain and Vk8 light chain express the AM14 BCR. To generate AM14 B cells that are deficient in TLR9, TLR7 or FcγRIIB, the AM14 and Vk8 genes were bred to the appropriate knock out mice.

### Example 2.2

### Antibodies and Reagents

All antibodies are mIgG2a unless indicated otherwise. The α-DNA reactive antibody PA4 was generously provided by Dr. M. Monestier (Temple University, Philadelphia, Pa.). The rat α-mouse IgM (mIgM) hybridoma B7-6 was generously provided by Dr. M. Julius (Sunnybrook Research Institute, Toronto, Canada). The mouse anti-chromatin IgG2a antibody PL2-3 was obtained from Dr. M. Monestier (Temple University, Philadelphia, Pa.). B cell survival factor BLyS was obtained from Human Genome Sciences. The experimental ligand for TLR9 (1826) was purchased from Invivogen (San Diego, Calif.). Prolong® Gold Antifade was obtained from Life Technologies (Carlsbad, Calif.). The anti-mouse IRF-4 antibody was obtained from Santa Cruz Biotechnology (Dallas, Tex.). The anti-goat IgG Alexa Fluor 647 was obtained from Jackson ImmunoResearch Laboratories (West Grove, Pa.). The DNA stain Sytox Blue was obtained from Life Technologies (Carlsbad, Calif.). Antinuclear antibody (ANA) reactivity was tested using an ANA test kit obtained from Antibodies Inc. (Davis, Calif.). The ANA test kit contains slides with wells that are coated with fixed HepG2 cells, a hepatocellular carcinoma cell line. Carboxyfluorescein diacetate, succinimidyl ester (CFSE) and TOP-RO-3 were obtained from Life Technologies (Carlsbad, Calif.). IRAK2 knock out mice (IRAK2KO) are described in Wan et al. (2009) J. Biol. Chem. 284: 10367-10375. IRAK4 knock in mice (IRAK2KO) are described in Kawagoe et al. (2007) J. Exp. Med. 204:1013-24 and were provided by Dr. X. L1 (Cleveland Clinic). Unless otherwise indicated, all secondary reagents were purchased from Jackson Immuno Research Laboratories (West Grove, Pa.).

### Example 2.3

### Cloning of Antibody Genes

The V regions of the IgM specific antibody B7-6 heavy and light chains were amplified by 5′ RACE according to standard methods. The antibody genes were verified for the absence of premature stop codons via sequencing (Ruberti et al. (1994) J. Immunol. Meth. 173(1): 33-9). The V regions of the nucleic acid specific antibody PA4 heavy and light chains were amplified by 5′ RACE according to standard methods. The specificities of the isolated antibody genes were confirmed by ANA test for PA4 and by ELISA for B7-6 and HB.

### Example 2.4

### Antinuclear Antibody (ANA) Test

The cloned BWR4 and PA4 antibodies as well as the DVD-Ig proteins were tested for ANA reactivity using the ANA test kit. Briefly, the DVD-Ig proteins and antibodies were diluted to 1 μg/ml in blocking buffer (1% BSA in PBS). The DVD-Ig proteins and antibodies were added to separate wells of an ANA slide at 50 μl/well and incubated in a moist chamber for 2 hours at room temperature (RT). The wells were then washed 3× with PBS. To detect bound antibodies, Alexa Fluor 488 conjugated goat α-human IgG (α-hIgG) was then added at a dilution of 1:1,000 and the slide incubated in a moist chamber for 1 hour. Unbound detecting antibody was removed by immersing the slide in a jar containing PBS and incubating for 10 minutes at RT with gentle rocking. The prior step was repeated 2× and 10 μl of Prolong® Gold Antifade was added to each well and a 24 mm×60 mm #1 cover glass seated on top. The covered slide was incubated overnight at RT and then sealed by applying clear nail polish to the edges. Binding of the DVD-Ig proteins and antibodies to the slides was assessed by fluorescence microscopy.

### Example 2.5

### IgM Binding ELISA

Reactivity of the DVD-Ig proteins with mIgM was determined by enzyme-linked immunosorbent assay (ELISA). Briefly, each well of an Ultra Cruz 96 well microtiter plate (Santa Cruz Biotechnology, Dallas, Tex.) was coated with mIgM at a concentration of 1 μg/ml in 100 μl of PBS. The plate was covered in plastic wrap and incubated overnight at 4° C. Each well of the plate was then washed 3× with 300 μl of PBST. After the final wash, 200 μl of blocking buffer (1% BSA in PBS) was added to each well and the plate incubated at RT for 2 hours. The plate was then washed as above. Rows B-H of the microtiter plate were filled with 100 μl of dilution buffer (1% BSA in PBST). The DVD-Ig proteins were then diluted to 1 μg/ml in dilution buffer and 147 μl of a DVD-Ig protein was added in duplicate to row A of a 96 well plate. For a ½ log dilution, 47 μl was transferred from row A to row B. The pipette tips were replaced with clean tips and 47 μl from row B was transferred to row C. The serial transfers were repeated as above until row G. To assess the binding of the secondary detection reagent to the coated IgM, the final transfer 47 μl was only transferred from G1-G10 to H1-H10 and wells H11 and H12 were left containing 100 μl of dilution buffer. To maintain a 100 μl assay volume, 47 μl was removed from wells H1-H10 and G11-G12 and discarded. After the serial dilution was completed, the plate was incubated for 1-2 hours at RT. The plate was washed as above and then 100 μl of horseradish peroxidase conjugated goat α-hIgG Fc specific, diluted 1:3,000 in dilution buffer, was added to each well. The plate was incubated for 1 hour at RT and then washed 4× as above. The plate was developed by adding 100 μl of 3′,3′,5′,5′-Tetramethylbenzidine (TMB) substrate solution to each well and incubating the plate in the dark for 10-15 minutes. The enzymatic reaction was stopped by adding 100 μl of 1M H₂SO₄ to each well. The plate was read at 450 nm on an EnVision 2102 multilabel reader (Perkin Elmer, Waltham, Mass.).

### Example 2.6

### Culture of Murine B Cells, BCR/TLR9 Stimulation, and [3H]thymidine Assay

Primary murine B cells were purified by positive selection using BD Imag™ CD45R/B220 magnetic particles (BD Biosciences, San Jose, Calif.). Briefly, B cells were cultured in RPMI containing, pencillin-streptomycin, β-mercaptoethanol, and 5% heat-inactivated fetal calf serum (FCS) in 96 well flat bottom plates at a density of 4×10⁵ cells/well. For stimulation through the BCR and TLR9, B cells were treated with a titration of 0.3 μg/ml-0.03 μg/ml of PA4 or 5 μg/ml to 0.15 μg/ml of a DVD-Ig protein (DVD3751, DVD3752, DVD3754, DVD3755, DVD3759 and DVD3760), unless otherwise noted. Cells were also treated with 1 μg/ml 1826, an experimental ligand for TLR9, as a control. After 24 hours, the cells were pulsed for 6 hours with [3H]thymidine (Perkin Elmer, Waltham Mass.). Incorporation of [3H]thymidine was quantified via a liquid scintillation beta counter (Trilux 1450 MicroBeta, PerkinElmer) according to standard methods.

### Example 2.6

### TLR9 Activation Assays and Flow Cytometry

Primary murine B cells were purified by positive selection as described above. For cultures >48 hours old, proliferation was quantified by carboxyfluorescein diacetate, succinimidyl ester (CFSE) dilution and cell death by TO-PRO-3 binding. To label the cells with CFSE, the cells were first washed 2 times with PBS. Next, the stock of CFSE was diluted to 10 μM in PBS and the cells were brought to 1×10⁷ cells/ml. The cells were mixed at a 1:1 ratio with 10 μM CFSE and mixed gently for 2 minutes. The CFSE labeling reaction was stopped by adding heat inactivated fetal calf serum equal in volume to the starting volume for the cell suspension. The cells were washed two times with media and resuspended to 1-4×10⁶ cells/ml and were incubated with 0.5 μg/ml DVD-Ig proteins (DVD3759 and DVD3754) or antibodies, with or with out 0.05 μg/ml Blys. Proliferation was measured by fluorophore dilution at 60-72 hours by flow cytometry. Briefly, the cells were pelleted and resuspended in FACS buffer (PBS, 3% FCS) containing 500 nM TO-PRO-3. Alternatively, the cells were resuspended in FACS buffer containing Sytox blue (1 μM). Both TO-PRO-3 and Sytox blue labeled dead cells by selectively staining the DNA of the cells with compromised plasma membranes. The only functional difference between TO-PRO-3 and Sytox blue are the excitation and emission spectra of the two fluorophores. For example, after 60-72 hrs, the cells were pelleted and resuspended in FACS buffer (PBS, 3% FCS) containing 500 nM TO-PRO-3. Analysis of proliferation and cell death were performed by FACS. Flow cytometric analysis was carried out using a BD LSR II with Diva Software (BD).

### Example 2.8

### Characterization of DVD-Ig Proteins that Bind Mouse IgM and DNA

DVD-Ig proteins DVD3751-DVD3760 were characterized by IgM binding assay at half-log dilution starting at 10 μg/ml-0.01 μg/ml according to the method of Example 2.5. DVD3756, DVD3757, DVD3759, and DVD3760 demonstrated the highest potency in that assay. The results are shown in Table 4.

DVD-Ig proteins DVD3751-DVD3760 were characterized by antinuclear antibody (ANA) staining at 10 μg/ml according to the method of Example 2.4. DVD3752, DVD3754, and DVD3755 demonstrated the highest level of staining. The results are shown in Table 4.

DVD-Ig proteins DVD3751-DVD3760 were characterized by ³H incorporation at 5 mg/ml according to the method of Example 2.6. DVD3751 and DVD3754 demonstrated the highest level of ³H incorporation. The results are shown in Table 4.

TABLE 4
Characterization of DVD-Ig Proteins that Bind Mouse IgM and DNA

| Parent | N-terminal | C-terminal | | IgM Binding | | ³H incorporation | |
|---|---|---|---|---|---|---|---|
| Antibody or | Variable | Variable | | EC50 | ANA | AM14 | AM14 |
| DVD-Ig ID | domain (VI) | domain (V2) | Linker | (μg/ml) | score | WT | TLR9−/− |
| DVD3751 | DNA | mIgM | GS | 0.1909 | 2 | (35554) | (1184) |
| | (PA4) | (B7.6) | | (X) | (X) | (XXX) | |
| DVD3752 | DNA | mIgM | SS | 0.205 | 4 | (12682) | (512) |
| | (PA4) | (B7.6) | | (X) | (XXX) | (XX) | |
| DVD3753 | DNA | mIgM | LL | — | — | — | — |
| | (PA4) | (B7.6) | | | | | |
| DVD3754 | DNA | mIgM | SL | 0.1167 | 4.5 | (35214) | (1597) |
| | (PA4) | (B7.6) | | (XX) | (XXX) | (XXX) | |
| DVD3755 | DNA | mIgM | LS | 0.09709 | 4.5 | (8498) | (402) |
| | (PA4) | (B7.6) | | (XX) | (XXX) | (X) | |
| DVD3756 | mIgM | DNA | GS | 0.0331 | 1 | (7793) | (459) |
| | (B7.6) | (PA4) | | (XXX) | | (X) | |
| DVD3757 | mIgM | DNA | SS | 0.04178 | 1 | (4063) | (382) |
| | (B7.6) | (PA4) | | (XXX) | | (X) | |
| DVD3758 | mIgM | DNA | LL | — | — | — | — |
| | (B7.6) | (PA4) | | | | | |
| DVD3759 | mIgM | DNA | SL | 0.04116 | 3.5 | (18554) | (970) |
| | (B7.6) | (PA4) | | (XXX) | (XX) | (XX) | |
| DVD3760 | mIgM | DNA | LS | 0.03529 | 1 | (4335) | (302) |
| | (B7.6) | (PA4) | | (XXX) | | (X) | |

(X)-(XXX) indicates relative results

### Example 2.9

### Characterization of DVD-Ig Proteins that Bind Human IgM and DNA

DVD-Ig proteins DVD3761, DVD3762, DVD3765, DVD3767, DVD3769, and DVD3770 were characterized by IgM binding assay at half-log dilution starting at 10 μg/ml-0.01 μg/ml essentially according to the method of Example 2.5. DVD3766, DVD3767, and DVD3769, demonstrated the highest potency in that assay. The results are shown in Table 5.

TABLE 5
Characterization of DVD-Ig Proteins that Bind Human IgM and DNA

| DVD | V1 Domain | V2 Domain | Linker | EC50 |
|---|---|---|---|---|
| DVD3761 | Anti-DNA | Anti-hIgM | GS | 0.5649 |
| DVD3762 | Anti-DNA | Anti-hIgM | SS | 1.863 |
| DVD3765 | Anti-DNA | Anti-hIgM | LS | 0.7987 |
| DVD3766 | Anti-hIgM | Anti-DNA | GS | 0.02112 |
| DVD3767 | Anti-hIgM | Anti-DNA | SS | 0.0292 |
| DVD3769 | Anti-hIgM | Anti-DNA | SL | 0.02579 |
| DVD3770 | Anti-hIgM | Anti-DNA | LS | 0.06843 |

DVD-Ig proteins specific for DNA and human IgM, and the parental IgG antibodies for these DVD-Ig proteins, were tested for ANA reactivity essentially as described in Example 2.4. From these experiments, it was determined that the DVD-Ig proteins exhibited the same ANA staining profile as the parental IgG antibodies.

### Example 3

### DVD-Ig™ Proteins that Bind Mouse IgM and DNA Induce Cell Proliferation and Cell Death in Primary Murine B Cells

Primary murine B cells were labeled and prepared as in Example 2.7 and stimulated with either media alone, 1 mg/ml of TLR9 ligand CpG oligodeoxynucleotide (ODN) 1826, 1 mg/ml of the mouse anti-chromatin autoantibody PL2-3 or 0.5 mg/ml DVD3759 in the presence or absence of 0.05 mg/ml of the B cell survival factor BLyS. After 72 hours, cells were stained with 1 μM of the cell permeable DNA stain Sytox Blue and analyzed by fluorescence activated cell sorting (FACS). Cell division was determined by dilution of CFSE and dead cells were identified by staining with Sytox Blue.

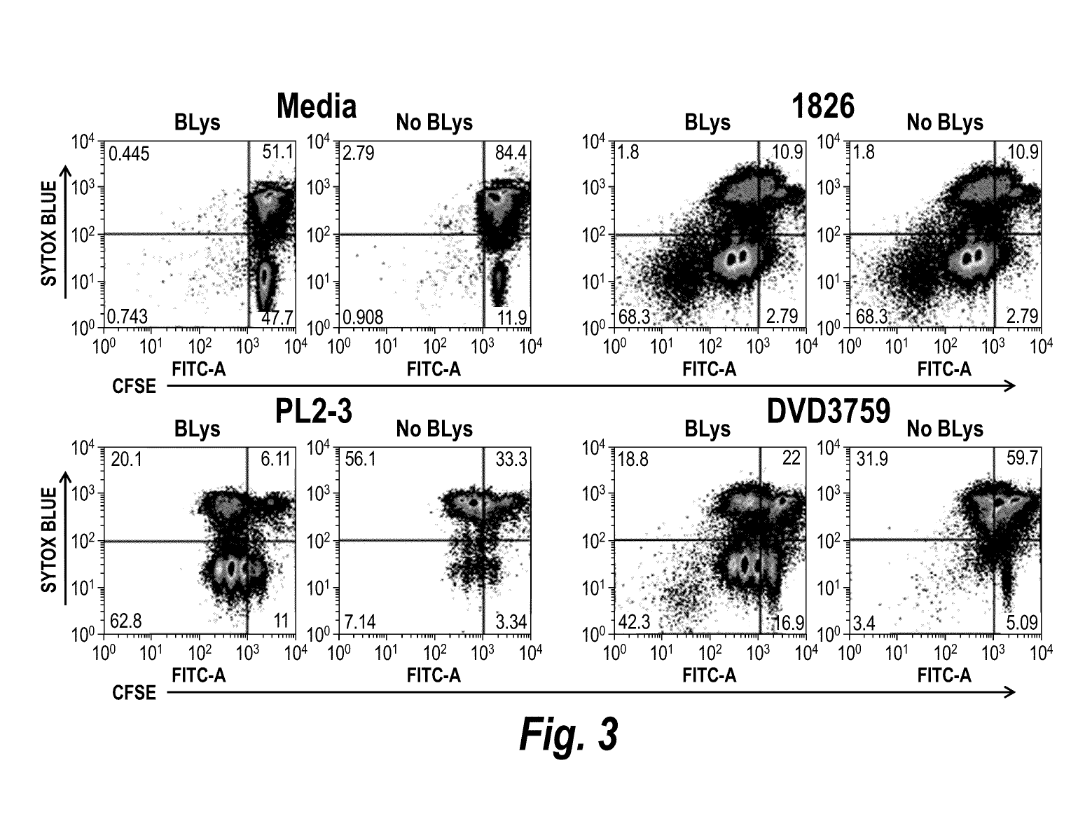

FIG. 3 shows that DVD3759 IgM/TLR9 co-engagement of the B cells induced several rounds of division followed by post-proliferative cell death. Cells could be rescued by the addition of BLyS. This post-proliferative cell death was not observed in cells stimulated with the TLR9 ligand 1826 or in the presence of the anti-chromatin autoantibody PL2-3.

### Example 4

### The DVD3759 Response is Dependent on IRAK4 Kinase Activity

To determine if IRAK4 kinase activity is required for activation and/or death induced TLR9 via DVD3759, either wild type (WT), IRAK2 knock out (IRAK2KO) or IRAK 4 knock-in (IRAK4 KI) primary B cells were purified and tested in a method similar to Example 2.7. Briefly, CFSE labelled B cells were plated at 4×10⁶ in a 96 well flat bottom palte incubated with either 1 μg/ml of CpG oligodeoxynucleotide (ODN) 1826 or 0.5 μg/ml of DVD3759, in the presence or absence of 0.05 μg/ml of the B cell survival factor BLyS. After 72 hours, cells were stained with 500 nM of the cell permeable DNA stain TOP-RO-3 and analyzed by FACS. Cell division was determined by dilution of CFSE and dead cells were identified by staining with TOP-RO-3.

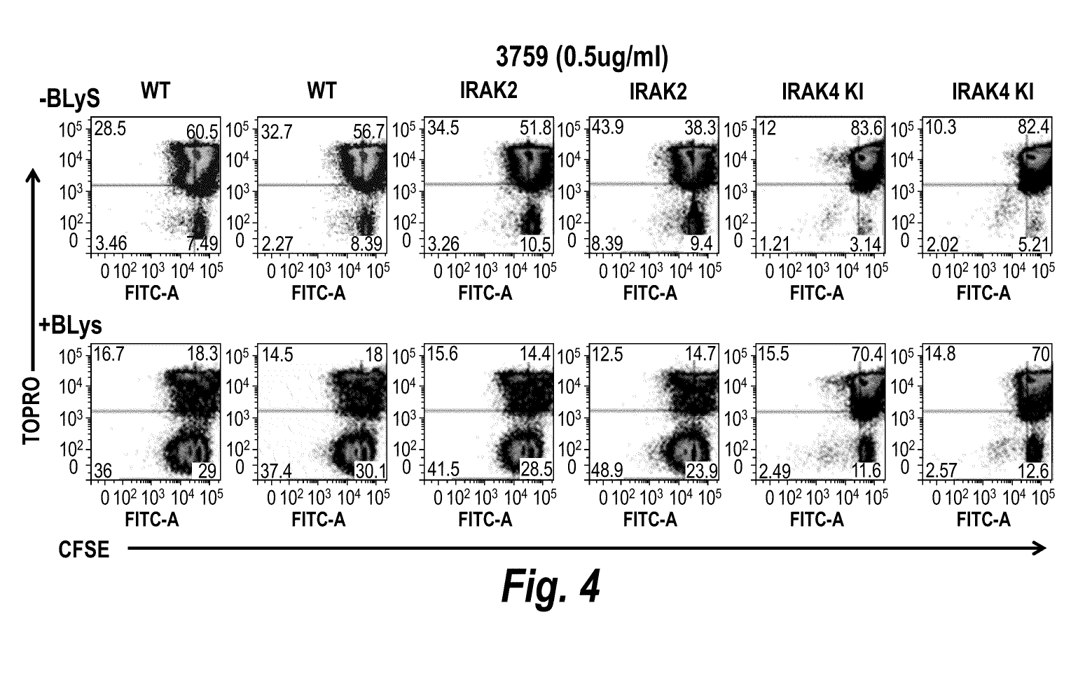

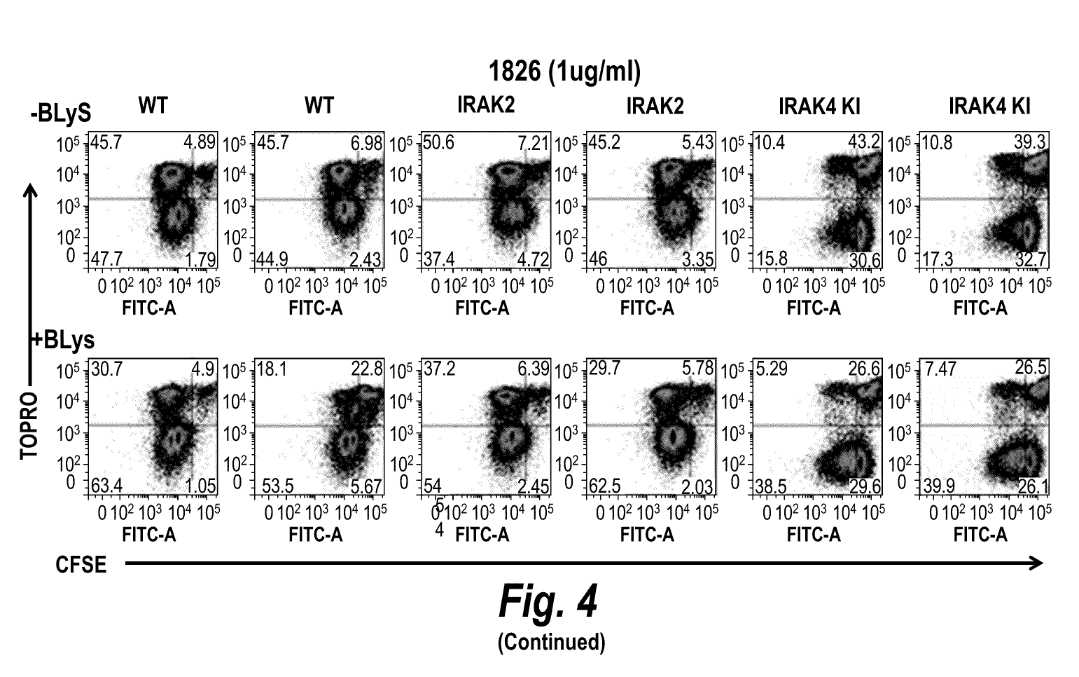

FIG. 4 shows that DVD3759 IgM/TLR9 co-engagement of the B cells induced several rounds of division followed by post-proliferative cell death. Cells could be rescued by the addition of BLyS. However, the DVD3759 post-proliferative cell death was not observed in IRAK4 KI B cells, indicating that IRAK4 kinase activity is required for the TLR9 dependent post-proliferative cell death induced by DVD3759.

### Example 5

### BCR/TLR9 Stimulation of Primary Human B Cells by DVD-Ig™ Proteins

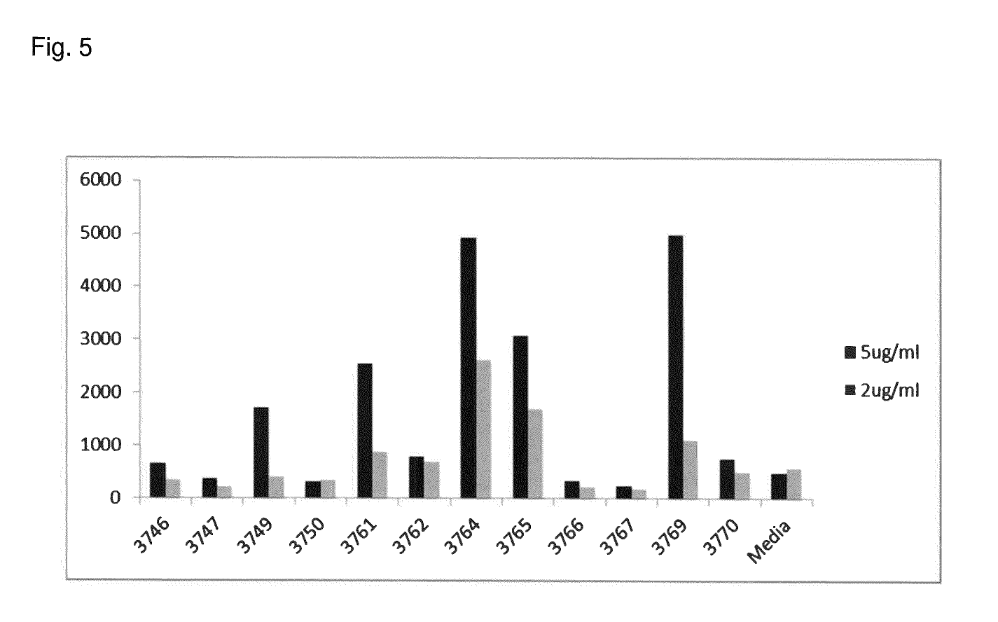

Primary human B cells were purified from 100 ml of blood drawn from a healthy donor. Peripheral blood mononuclear cells (PBMCs) were purified using Ficoll separation and depleted of CD3+ cells by negative selection. The CD3 depleted fraction was used to purify CD19+CD27− naïve B cells using the EasySep™ Human Naïve B Cell Enrichment kit (Stemcell Technologies, Vancouver, Canada). Briefly, B cells were cultured in RPMI containing, pencillin-streptomycin, β-mercaptoethanol, and 10% heat-inactivated fetal calf serum (FCS) in 96-well round bottom plates at a density of 1×10⁵ cells/well. For stimulation through the BCR and TLR, B cells were treated with 1 μg/ml or 5 μg/ml of a DVD-Ig protein (DVD3746, DVD3747, DVD3749, DVD3750, DVD3761, DVD3762, DVD3764, DVD3765, DVD3766, DVD3767, DVD3769 and DVD3770), or media unless otherwise noted. After 48 hours, the cells were pulsed for 12 hours with [3H]thymidine (Perkin Elmer, Waltham Mass.). Incorporation of [3H]thymidine was quantified via a liquid scintillation beta counter (Trilux 1450 MicroBeta, PerkinElmer) according to standard methods. The results of the proliferation assays are set forth in FIG. 5.

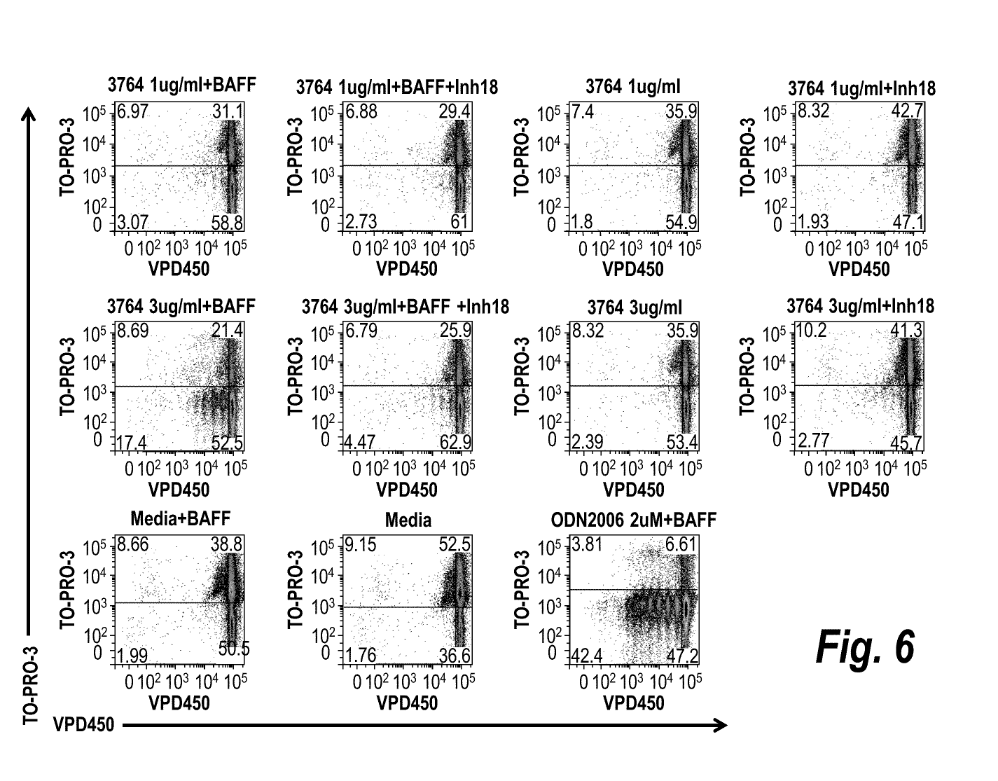

In a further set of experiments, primary human B cells were purified as described above. For cultures greater than 108 hours old, proliferation was quantified by Violet Proliferation Dye 450 (VPD450) dilution and cell death by TO-PRO-3 binding. To label the cells with VPD450, the cells were washed twice with PBS and brought to 1×10⁷ cells/ml in PBS. VPD450 was then added to a final concentration of 3.5 μM, and the cells were mixed and incubated for 5 minutes in a 37° C. water bath. The labeling reaction was stopped by adding ice-cold RPMI medium. The cells were washed twice with media, resuspended to 1×10⁶ cells/ml, and incubated with 1 μg/ml or 3 μg/ml of DVD-Ig protein (DVD3764) with or without TLR9 inhibitor 18 (Inh18), or 2 μM of ODN2006 (Invivogen, San Diego, Calif.), with or with out 500 ng/ml B-cell activating factor (BAFF). Proliferation was measured by fluorophore dilution at 108 hours by flow cytometry. Briefly, the cells were pelleted and resuspended in FACS buffer (PBS, 3% FCS) containing 500 nM TO-PRO-3. TO-PRO-3 labeled dead cells by selectively staining the DNA of the cells with compromised plasma membranes. Analysis of proliferation and cell death were performed by FACS. Flow cytometric analysis was carried out using a BD LSR II with Diva Software (BD). The results of the flow cytometric analysis are set forth in FIG. 6.

These data demonstrate that DVD-Ig molecules that specifically bind to human IgM and DNA can stimulate primary human B cells to proliferate in a TLR9-dependent manner

### Example 6

### TLR-Dependent Activation of Autoreactive B Cells in DNase II-Deficient Mice

The inability to express both the phagolysosomal endonuclease, DNase II, and the receptor for type I IFN, IFNaR1, leads to the development of inflammatory arthritis, through a mechanism dependent on the adaptor of cytosolic DNA sensors, STING. These double knockout (DKO) mice also develop other indications of systemic autoimmunity, including production of anti-nuclear autoantibodies (ANA) and splenomegaly. Immunoflourescent staining patterns revealed that autoantibodies in DKO mice are predominantly directed against RNA-associated autoantigens, commonly targeted in TLR7-dominated SLE-prone mice, and not DNA-associated autoantigens, as might be expected from the accrual of excessive levels of undegraded DNA. Unc93b1^{-/-} DKO mice do not produce ANA, or develop splenomegaly, and therefore endosomal TLRs must play a key role in the immune activation characteristic of DKO mice. To further explore the role of TLR9 in autoantibody production, we developed bifunctional binding proteins specific for IgM and DNA that allowed us to direct DNA complexes to non-Tg B cells. These IgM/DNA DVD-Ig™ molecules activate B cells through a TLR9-dependent mechanism. DKO B cells failed to respond to the IgM/DNA DVD-Ig™ molecule, despite a normal response to both CpG ODNs and anti-IgM. Thus in the absence of DNaseII, B cells cannot respond to DNA-associated autoantigens.

### Introduction

DNase II is a lysosomal endonuclease that plays a critical role in the degradation of the extracellular DNA debris generated by homeostatic erythropoiesis and apoptosis. In mice, DNase II deficiency leads to the overproduction of type I IFN and results in an embryonically lethal anemia (Yoshida et al. (2005) Nature Immunol. 6:49-56). Dnase2^{-/-} Ifnar1^{-/-} mice that do not express a functional type I IFN receptor survive to adulthood but then develop a form of inflammatory arthritis associated with the production of anti-nuclear antibodies (ANAs) (Kawane et al. (2006) Nature 443:998-1002). Both embryonic lethality and arthritis appear to depend on cytosolic DNA sensors that converge on the adaptor molecule STING, as STING-deficient (Tmem23^{-/-}) Dnase2^{-/-} mice survive to adulthood without evidence of arthritis (Ahn et al. (2012) Natl. Acad. Sci. USA 109:19386-19391).

It has been proposed that autoantibody production depends on the adjuvant-like activity of autoantigens (Leadbetter et al. (2002) Nature 416:603-607; Busconi et al. (2006) J. Endotoxin Res. 12:379-384; Plotz (2003) Nat. Rev. Immunol. 3:73-78). In the context of SLE and related diseases, detection of endogenous nucleic acid-associated autoantigens by B cell endosomal Toll-like receptors is required for the production of ANAs (Christensen et al. (2006) Immunity 25:417-428; Jackson et al. (2014) J. Immunol. 192:4525-4532; Kono et al. (2009) Proc. Natl. Acad. Sci. USA 106:12061-12066; Lau et al. (2005) J. Exp. Med. 202:1171-1177; Leadbetter et al. (2002) Nature 416:603-607). The same TLRs also play a role in the activation of dendritic cells, and neutrophils (Boule et al. (2004) J. Exp. Med. 199:1631-1640; Garcia-Romo et al. (2011) Sci. Transl. Med. 3:73ra20; Hua et al. (2014) J. Immunol. 192:875-885). However, neither the DNA sensor TLR9, nor the RNA sensor TLR7, is required for the development of arthritis in Dnase2−/− Ifnar1−/− mice (Kawane et al. (2010) Proc. Natl. Acad. Sci. USA 107:19432-19437), as mice that fail to express the critical TLR downstream signaling components, MyD88 and TRIF, still develop arthritis (Kawane et al., 2010). Intriguingly, Dnase2^{-/-} Ifnar1^{-/-} mice have also been reported to make autoantibodies, including autoantibodies against DNA (Kawane et al., 2006), while at least one report has suggested that the production of anti-DNA antibodies in this model requires STING, as Tmem23^{-/-} Dnase2^{-/-} mice fail to make anti-DNA autoantibodies (Aim et al., 2012). Nevertheless, our own preliminary autoantigen microarray data has indicated that Dnase2^{-/-} Ifnar1^{-/-} mice make antibodies against an extensive panel of nuclear autoantigens and that autoantibody production even in this model is dependent on the expression of Unc93B1 and therefore endosomal TLRs (Baum et al manuscript in preparation). To better understand the specificity of the autoantibodies produced by Dnase2^{-/-} Ifnar1^{-/-} mice, we have now screened sera for ANA reactivity patterns by immunofluorescent staining of HEp2 cells. We have also developed novel bifunctional autoantibodies to test the capacity of Dnase2^{-/-} Ifnar1^{-/-} B cells to respond to DNA ICs. We find that most of the anti-nuclear autoantibodies produced by these mice are directed against RNA-associated autoantigens and not dsDNA. Moreover the lack of anti-dsDNA autoantibodies correlates with the inability of Dnase2^{-/-} Ifnar1^{-/-} B cells to respond to DNA.

Mice: RF and Tlr-9^{-/-} mice have been described previously (Uccellini et al. (2008) J. Immunol. 181:5875-5884), The DNase II-deficient mice on a C57BL/6 background were kindly provided by Dr. S, Nagata and obtained from the RIKEN Institute. IFNγR1-deficient and Unc93B1-deficient mice were obtained from Jackson Lab. DnaseII^{-/-} Ifnar1^{-/-} (Het), DnaseII^{-/-} Ifnar1−/− (DKO), and DnaseII^{-/-} Unc93b1^{-/-} (TKO), were bred at UMMS. All mice were maintained at the Department of Animal Medicine of the University of Massachusetts Medical School in accordance with the regulations of the American Association for the Accreditation of Laboratory Animal Care.

DVD-Ig™ binding protein: DVD3754 comprises SEQ ID NO: 50 for the heavy chain and SEQ ID NO: 51 for the light chain. Methods for constructing DVD-Ig molecules are shown for example in U.S. Pat. No. 7,612,181 and Wu et al. 2009 mAbs 1:339-347, incorporated by reference, herein in their entireties. The variable heavy (VH) and variable light (VL) regions were PCR-cloned from hybridomas producing a mouse-anti-DNA mAb (PA4) (Monestier et al., Eur J Immunol. 1994 March; 24(3):723-30), incorporated by reference herein in its entirety, and a rat anti-mouse IgM mAb (B7-6) (Julius et al., Eur J Immunol. 1984 August; 14(8):753-7), incorporated by reference herein in its entirety, using oligo mixtures of NLH5/NLH3 for VH, oligo mixtures of NLK5/NLK3 for VL which are slightly modified primer sets based on Mouse Ig-Primer Set (Novagen, Cat#69831-3). The VH/VL PCR fragments were then subcloned into mammalian expression plasmids containing the human IgG1 constant region sequenced, expressed in HEK293-6E cells, purified using standard protein A, and physically (SEC, MS) and functionally characterized alongside hybridoma-derived mAbs. The VH and VL sequences of each mAb were then used to design DVD-Ig molecules as described previously (Wu et al., 2009). The DVD-Ig molecules were synthesized, subcloned into mammalian expression plasmids containing IgG1 constant region, expressed and purified to homogeneity for further characterization.

ANA: HEp-2 human tissue culture substrate slides were incubated with 5 μg/ml of the anti-DNA-IgG2a or the DVD-Ig™ binding protein for 2 hours at RT. The slides were washed and bound antibodies were detected with Alexa-488 goat anti-human antibody or DyLight 488 goat anti-mouse antibody.

IgM binding ELISA: Titrations of the DVD-Ig™ proteins (10 μg/ml to 0.01 μg/ml) were added to ELISA plates coated with murine IgM. Bound antibody was detected with biotinylated anti-human IgG and streptavidin-HRP. EC50 were calculated from the titration curve using Prism6.

Proliferation of B220+ B cells: B cells were purified by magnetic bead separation using CD45R/B220-conjugated magnetic particles (BD Biosciences, San Jose, Calif., USA) and stimulated as described previously (Nundel et al. (2013) J. Leukoc. Biol. 94:865-875) with 15 μg/ml goat anti-mouse IgM F(ab′)₂ (Jackson ImmunoResearch), ODN1826 (CpG; Idera Pharmaceuticals), anti-DNA-mAb (Leadbetter et al. (2002) Nature 416:603-607), or DVD-Ig proteins (1 μg/ml). B cell proliferation was assessed by ³H-thymidine (Amersham Biosciences, Piscataway, N.J., USA) incorporation at 24 hours post-stimulation. For long-term proliferation assays, B220-purified B cells were labeled with a final concentration of 2.5 μM CFSE (Life Technologies) in PBS for 2 minutes. The cells were then washed and cultured for 72 hours in the presence of BLyS (Human Genome Sciences).

FACS for B cells: Multicolor flow cytometry analysis was carried out using a BD LSR II with DIVA software (BD Biosciences). Analysis was conducted with FlowJo software (Tree Star, Ashland, Oreg., USA) Immature and mature B cell ratios were determined using Pacific Blue-B220 and APC-AA4.1 (eBioscience). Dead cells and debris were excluded by forward- and side-scatter. Proliferation was assessed by CFSE dilution. Cell death was ascertained using the DNA stain TO-PRO-3 (Life Technologies) at a final concentration of 20 nM.

### Results and Discussion

### Autoantibody Production in DNaseII^{-/-}×IFNaR2^{-/-} Double Knockout Mice is Endosomal

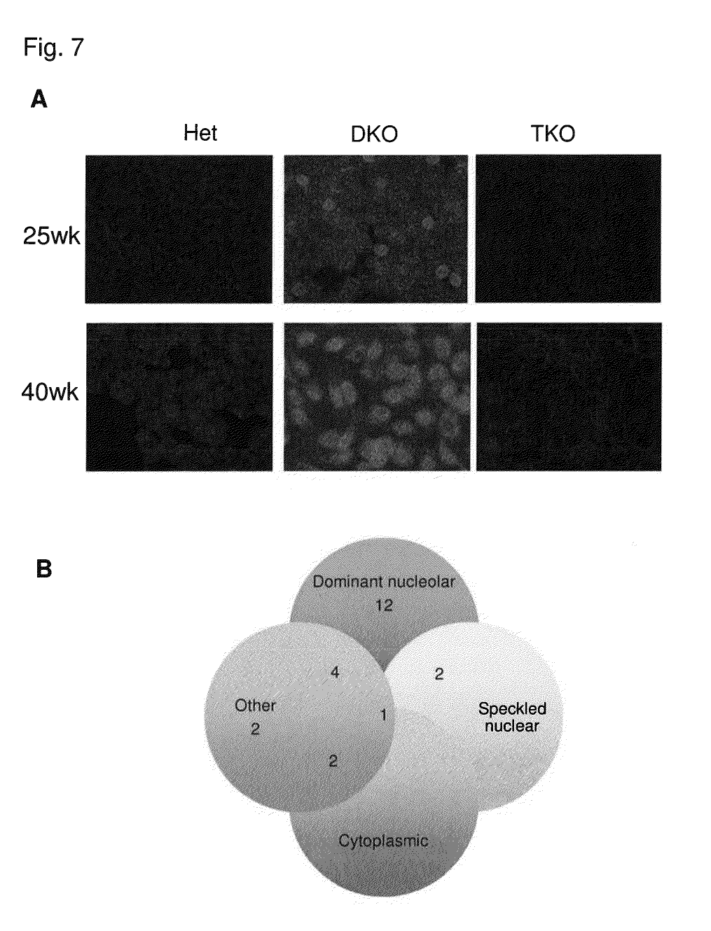

Dnase2^{-/-} Ifnar1^{-/-} double knockout (DKO) mice had previously been reported to make anti-DNA antibodies, as determined by solid phase ELISA (Kawane et al. (2006) Nature 443:998-1002). However, DNA is a highly charged molecule and direct binding assays can often detect relatively non-specific interactions. To better understand the autoantigen specificity of the autoantibodies produced by DKO mice, sera were collected at early (20-25 weeks) and later (>40 weeks) stages of the disease process, and evaluated by immunofluorescent staining of HEp-2 cells. It was expected that a homogeneous nuclear staining pattern associated with the delineation of mitotic plates, indicative of autoantibodies reactive with dsDNA or other chromatin components. While almost all DKO sera made ANAs, quite unexpectedly, almost 80% (18 out of 23) of the sera from early bleeds exhibited a prominent nucleolar staining pattern. The remainder of the early sera showed additional speckled nuclear, or cytoplasmic staining (FIG. 7A, 7B), but none of the sera stained mitotic plates. By contrast, all the sera from Dnase^{+/-} heterozygous (Het) Ifnar1^{-/-} mice were completely ANA-negative at early time points and only a limited number became very weakly positive at later time points. As the DKO mice aged, the staining patterns became more complex, indicative of epitope spreading, but still more likely to be categorized as speckled nuclear, rather than homogeneous nuclear (FIG. 7A); again, we did not find any sera that stained mitotic plates, as would be expected of autoantibodies reactive with dsDNA. Anti-nucleolar and/or anti-SmRNP antibodies are commonly found in SLE-prone mice where TLR7, an RNA-specific receptor, plays a prominent role (Bolland et al., 2002. Genetic modifiers of systemic lupus erythematosus in FcgammaRIIB(−/−) mice. J Exp Med 195:1167-1174). Overall, these results indicate that autoantibodies produced by Dnase2^{-/-} Ifnar1^{-/-} DKO mice predominantly recognize RNA-associated autoantigens.

While both TLR7 and TLR8, located in endolysosomal compartments, have been implicated in the detection of autoantibodies reactive with RNA-associated autoantigens (Lau et al. (2005) J. Exp. Med. 202:1171-1177; Pisitkun et al. (2006) Science 312:1669-1672; Subramanian et al. (2006) Proc. Natl. Acad. Sci. USA 103:9970-9975), RNA sensors such as RIG-I and MDA5 are present in the cytosol and could potentially also contribute to autoantibody production Unc93B1 is a chaperone protein required for the transport of nucleic acid sensing TLRs to the lysosomal compartment; in the absence of functional Unc93B1, mice fail to respond to all TLR7, TLR8 and TLR9 ligands (Tabeta et al. (2006) Nature Immunol. 7:156-164). Remarkably, Dnase2^{-/-} Ifnar1^{-/-} Unc93b1^{-/-} triple knockout (TKO) mice fail to make ANAs, as determined both by immunofluorescence staining (FIG. 7A) and by autoantigen microarrays. Therefore, even in a STING-dependent arthritis model, endosomal TLRs are absolutely required for the generation of ANAs directed against RNA-associated autoantigens.

It was interesting that there was an RNA-autoantigen bias observed in a model of systemic autoimmunity triggered by excessive levels of DNA. Since the phagocytosis of extruded RBC nuclei and apoptotic debris most likely leads to the initial accumulation of nucleic acids in phagocytic compartments associated with endosomal TLRs, including TLR9 (Henault et al. (2012) Immunity 37:986-997), it was not surprising to find evidence of endosomal TLR activation. Nevertheless, given the excessive build-up of DNA, it was expected that there would be a more DNA-centric response and the production of ANAs with a homogeneous nuclear, rather than a nucleolar or speckled nuclear, staining pattern. The apparent absence of dsDNA-reactive antibodies pointed to a functional defect in the TLR9 signaling cascade.

### IgM/DNA DVD-Ig™ Molecules Target DNA ICs to Non-Transgenic B Cells

In the initial studies, it was found that Dnase2^{-/-} Ifnar1^{-/-} DKO B cells responded normally to small molecule CpG ODN-based TLR9 ligands. However, it was important to test these cells with more disease-relevant DNA-associated autoantigen complexes. B cells expressing a transgene-encoded low affinity BCR specific for autologous IgG2a can be activated by IgG2a DNA-reactive monoclonal autoantibodies, and not hapten-specific monoclonal antibodies, through a mechanism that is entirely dependent on TLR9 (Leadbetter et al. (2002) Nature 416:603-607). These rheumatoid factor (RF) B cells provide an experimental readout for examining the response of a prototypic autoreactive B cell responder population to spontaneously forming immune complexes (ICs). However, this approach is limited to cells expressing the correct BCR transgene and therefore the evaluation of multi-gene genetically targeted mice has required extensive intercrossing of the relevant strains to generate mice with the mutations of choice that also express the appropriate RF heavy and light chains (Nundel et al. (2013) J. Leukoc. Biol. 94:865-875).

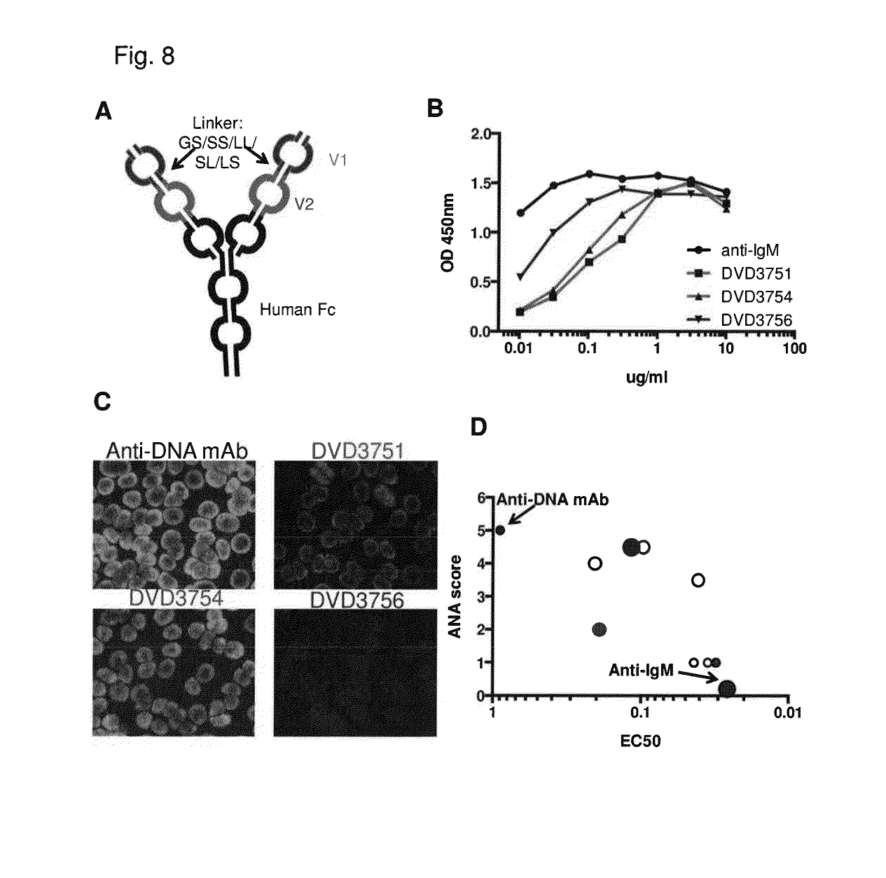

To expedite the analysis of gene targeted B cell responses to autoantigen ICs, bifunctional immunoglobulins were developed that incorporate both DNA and IgM binding domains, and therefore direct DNA-associated ICs to all IgM expressing B cells. The DNA binding domain used for the construction of these antibodies came from an IgG2a DNA-reactive monoclonal antibody, selected for its capacity to activate RF B cells through a TLR9-dependent mechanism. The platform we selected, DVD-Ig™ binding proteins, comprises conventional antibody heavy and light chains that incorporate the VL and VH domains of two antibodies, fused in tandem by a short linker, connected to human constant region domains, to essentially create 2 distinct variable domains in each Fab (Wu et al. (2007) Nature Biotechnol. 25:1290-1297)(FIG. 8A). DVD-Ig™ binding proteins can be constructed with either one of the binding domains at the N-terminus, and with linkers of distinct lengths between the two domains. The orientation and linker combination that allows for the optimal binding activity of both V domains can vary, depending on V domain combinations. To identify a DVD-Ig™ binding protein that expressed both IgM and DNA reactivity, 8 different DVD-Ig™ binding proteins with either the IgM or the DNA V domain at the N-terminus, connected with 5 different linkers, were evaluated for their capacity to bind IgM, as determined by a direct binding ELISA, and to bind DNA, as determined by an immunofluorescent HEp-2 staining assay. In general the DVD-Ig™ binding proteins with an N-terminal anti-IgM domain bound IgM with higher affinity, although internal anti-IgM domains were also functional (FIG. 8B). By contrast, only the DVD-Ig™ binding proteins with an N-terminal anti-DNA domain were positive by ANA, and the intensity of staining varied within this group based on the linker between the V1 and V2 domains (FIG. 8C). DVD-Ig™ binding proteins reactive with IgM and/or DNA were assayed for their capacity to activate B cells and the relative level of response for representative DVDs was assessed by 3H-thymidine incorporation (FIG. 8D). One particular DVD-Ig™ binding protein, DVD3754, with a high ANA score and intermediate IgM binding affinity, stimulated BALB/c B cells more strongly than the rest.

### IgM/DNA DVD-Ig™ Binding Protein Activation of B Cells is TLR9 Dependent

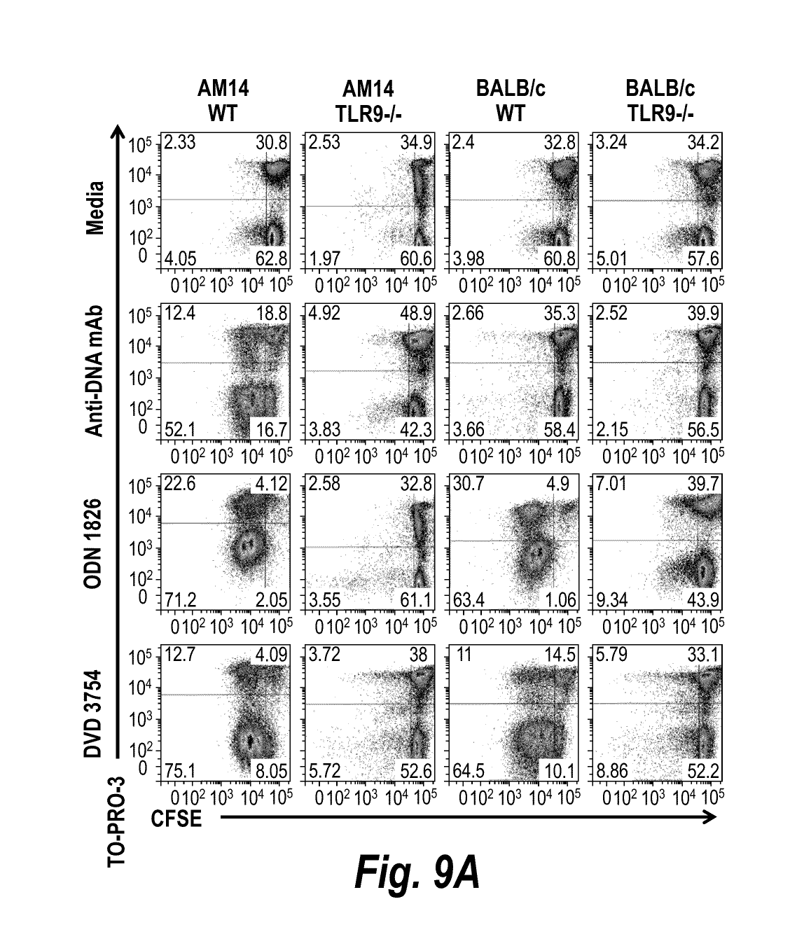

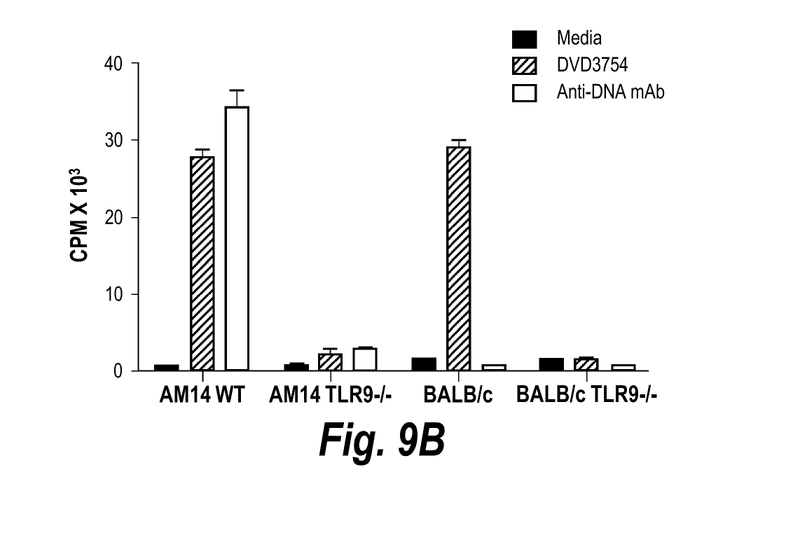

The capacity of IgG2a dsDNA-reactive autoantibodies to activate RF B cells depends on endogenous DNA, presumably released from damaged or dying cells. Although the actual ligand in this assay system is not defined, the RF B cell response has been shown to be markedly decreased in the presence of exogenous DNaseI, and Tlr9^{-/-} RF B cells cannot respond to IgG2a DNA-specific mAbs (Leadbetter et al. (2002) Nature 416:603-607). To further characterize the IgM/DNA DVD-Ig™ binding proteins, they were directly compared to the original IgG2a anti-DNA mAb for their ability to activate both Tlr9^{+/+} and Tlr9^{-/-} RF Tg and non-Tg B cells. As found previously, the anti-DNA mAb only activated Tlr9^{+/+} RF B cells. By contrast, DVD3754 induced both RF and non-Tg BALB/c B cells, but not RF Tlr9^{-/-} or BALB/c Tlr9^{-/-} B cells, to proliferate, and the level of activation was comparable to the anti-DNA mAb (FIG. 9A,B). Therefore, IgM/DNA DVD3754 activation of polyclonal B cells is TLR9-dependent and recapitulates the mechanism through which anti-DNA mAbs activate RF B cells. IgM/DNA DVD3754 can therefore be used to interrogate the DNA responses of additional BCR non-Tg gene-targeted strains.

### DVD3754 IC Activation of B Cells Requires DNase II

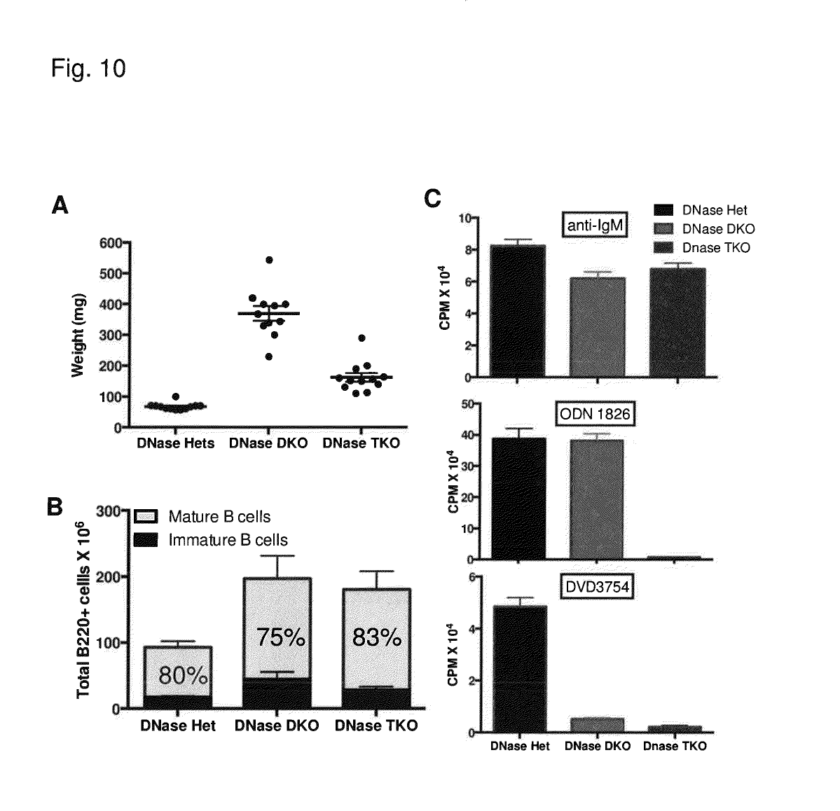

Apart from arthritis, Dnase2^{-/-} Ifnar1^{-/-} mice develop extensive splenomegaly even at a very early age. This splenomegaly has been attributed to extramedullary hematopoiesis resulting from suboptimal generation of mature RBCs in the bone marrow. During related to the inability of bone marrow macrophages to degrade RBC nuclei generated during the final stages of RBC differentiation (Kawane et al. (2006) Nature 443:998-1002). In contrast to arthritis, splenomegaly is not triggered by a STING-dependent pathway as Dnase2^{-/-} Tmem23^{-/-} mice still develop splenomegaly. However, splenomegaly was greatly reduced in the Dnase2^{-/-} Ifnar1^{-/-} Unc93b1^{-/-} TKO mice, even though these mice should have a comparable problem in the clearance of cell debris, and therefore a requirement for extramedullary hematopoiesis (FIG. 10A). To assure that both the DKO and TKO spleens, despite their different sizes, contained comparable mature follicular B cell compartments, DKO and TKO splenic B cells were compared to Dnase2^{+/-} Ifnar1^{-/-} B cells by flow cytometry for expression of B220 and AA4.1. All three strains contained relatively comparable ratios of mature and immature B cells (FIG. 10B) and these B cells failed to express B cell activation markers such as CD69 or CD86 (data not shown). Therefore it was expected that they would respond comparably to BCR-directed ligands.

To then address the lack of dsDNA-specific autoantibodies in the DKO sera, heterozygous Dnase2^{+/-} Ifnar1^{-/-}, homozygous Dnase2^{-/-} Ifnar1^{-/-} DKO, and Dnase2^{-/-} Ifnar1^{-/-} Unc93b1^{-/-} TKO B cells were stimulated with anti-IgM F(ab′)₂, the small molecule TLR9 ligand ODN 1826 and DVD3754. As expected, mature B cells from all 3 strains responded comparably to anti-IgM, and the Dnase2^{-/-} Ifnar1^{-/-} Unc93b1^{-/-} TKO mice failed to respond to both the small molecule TLR ligands as well as DVD3754. Unexpectedly, the DKO B cells also failed to respond to DVD3754 (FIG. 10C), despite a normal response to ODN 1826. DNase II has previously been shown to play a critical role in engulfment-mediated DNA degradation (Kawane et al. (2001) Science 292:1546-1549), while in C. elegans, effective degradation of cell corpses requires expression of a DNase II homologue in both the original apoptotic cell, and in the phagocytic cell that engulfs the cell corpse (Evans and Aguilera (2003) Gene 322:1-15). The inability of DKO B cells to respond to DVD3754 points to a B cell intrinsic role for DNase II in the degradation and therefore detection of endogenous DNA fragments delivered through the BCR, thereby explaining the absence of antibodies reactive with dsDNA. Presumably, TLR9 is still functional in the DKO B cells because they still respond to ODN 1826, which does not require degradation. Excessive amounts of undegraded DNA presumably then interfere with the capacity of macrophages or other phagocytic cells to appropriately clear cell debris containing both RNA—as well, as RNA-associated autoantigens, thereby triggering the production of autoantibodies through a mechanism dependent on RNA-reactive TLRs.

Although TLR9 is required for the production of anti-dsDNA antibodies in autoimmune-prone mice, SLE-prone Tlr9^{-/-} mice invariably develop more severe clinical disease than their TLR9-sufficient littermates (Christensen et al. (2006) Immunity 25:417-428; Jackson et al. (2014) J. Immunol. 192:4525-4532; Yu et al. (2006) Int. Immunol. 18:1211-1219). It has been proposed that TLR9 is required for the production of protective antibodies that are important in the clearance of apoptotic or other forms of cell debris that serves as the trigger for systemic autoimmunity (Stoehr et al. (2011) J. Immunol. 187:2953-2965). Another possible explanation for the negative regulatory role of TLR9 is that TLR7 driven B cell responses are inherently limited by the co-expression of TLR9-dependent autoantibodies directed at RNA-associated autoantigens are simply more pathogenic due to distinct activation pathways, or other unique properties of antibodies directed to the RNA-associated autoantigens. The inability of Dnase2^{-/-} Ifnar1^{-/-} DKO mice to respond to endogenous TLR9 ligands adds this model to the list of predominantly RNA-driven TLR-dependent systemic autoimmune diseases.

Although the development of arthritis in DKO mice is TLR-independent (Kawane et al. (2010) Proc. Natl. Acad. Sci. USA 107:19432-19437), our data implicate B cell expression of TLR7 in autoantibody production. Intriguingly, splenomegaly was dramatically reduced in Dnase2^{-/-} Ifnar1^{-/-} Unc93b1^{-/-} TKO mice. Additional intercrosses will be needed to determine whether TLR7, or other Unc93B1-dependent TLRs, are responsible for the non-arthritic hematopoietic disorders exhibited by DKO mice. In either case, the current study demonstrated that the autoimmune and inflammatory features of Dnase2^{-/-} Ifnar1^{-/-} DKO require TLRs other than TLR9.

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Ausubel et al. (eds.), CURRENT PROTOCOLS IN MOLECULAR BIOLOGY, John Wiley &Sons, NY (1993);
Ausubel, F. M. et al. eds., SHORT PROTOCOLS IN MOLECULAR BIOLOGY (4th Ed. 1999) John Wiley & Sons, NY. (ISBN 0-471-32938-X);
CONTROLLED DRUG BIOAVAILABILITY, DRUG PRODUCT DESIGN AND PERFORMANCE, Smolen and Ball (eds.), Wiley, New York (1984);
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, N.Y., (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, US Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).
Kriegler, Gene Transfer and Expression, A Laboratory Manual, Stockton Press, NY (1990);
Lu and Weiner eds., CLONING AND EXPRESSION VECTORS FOR GENE FUNCTION ANALYSIS (2001) BioTechniques Press. Westborough, Mass. 298 pp. (ISBN 1-881299-21-X).
MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION TO GENETIC ENGINEERING (3d Ed. 1985) Blackwell Scientific Publications, Boston. Studies in Microbiology; V.2:409 pp. (ISBN 0-632-01318-4).
Sambrook, J. et al. eds., MOLECULAR CLONING: A LABORATORY MANUAL (2d Ed. 1989)
Cold Spring Harbor Laboratory Press, NY. Vols. 1-3. (ISBN 0-87969-309-6).
SUSTAINED AND CONTROLLED RELEASE DRUG DELIVERY SYSTEMS, J. R. Robinson, ed., Marcel Dekker, Inc., New York, 1978
Winnacker, E. L. FROM GENES TO CLONES: INTRODUCTION TO GENE TECHNOLOGY (1987) VCH Publishers, NY (translated by Horst Ibelgaufts). 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A bispecific binding protein that binds to at least two targets, wherein target one comprises a TLR-signaling autoantigen and target two comprises an immune cell receptor.

2. The bispecific binding protein of claim 1, wherein the TLR-signaling autoantigen comprises a nucleic acid selected from the group consisting of a deoxyribonucleic acid (DNA) and a ribonucleic acid (RNA).

3. The bispecific binding protein of claim 1, wherein the immune cell receptor comprises a surface bound immunoglobulin or fragment thereof.

4. The bispecific binding protein of claim 1, wherein the immune cell receptor is a B cell receptor (BCR).

5. The bispecific binding protein of claim 1, wherein the immune cell is a B cell.

6. The bispecific binding protein of claim 1, wherein the TLR comprises TLR7 or TLR9.

7. The bispecific binding protein of claim 1, wherein target two comprises an IgM immunoglobulin.

8. The bispecific binding protein of claim 1, wherein target two comprises an immunoglobulin selected from the group consisting of an IgD, an IgE, an IgA, and an IgG.

9. The bispecific binding protein of claim 1, wherein target two comprises an allotypic or idiotypic immunoglobulin.

10. The bispecific binding protein of claim 1, wherein target two comprises an immunoglobulin light chain and/or an immunoglobulin heavy chain.

11. The bispecific binding protein of claim 1, wherein the bispecific binding protein causes cell proliferation and/or cell death.

12. The bispecific binding protein of claim 1, wherein the bispecific binding protein comprises a format selected from the group consisting of a DVD-Ig™ molecule, a BiTe® molecule, a DART® molecule, a DuoBody™ molecule, a scFv/diabody-IgG molecule, a cross-over multispecific molecule, a 2-in-1 bispecific molecule, a knob-in-hole multispecific molecule, a CovXBody molecule, an affibody molecule, a scFV/diabody-CH2/CH3 bispecific molecule, a IgG-non-Ig protein scaffold-based multispecific molecule, a Fynomer® molecule and a scFV/diabody linked to normal human protein like human serum albumin-bispecific molecule.

13. The bispecific binding protein of claim 1, wherein the bispecific binding protein comprises a first and a second polypeptide chain, each independently comprising the format VD1-(X1)n-VD2-C-(X2)n, wherein VD1 is a first variable domain, VD2 is a second variable domain, C is constant domain, X1 is a linker, X2 is an Fc region, and n is 0 or 1, wherein the VD1 domains on the first and second polypeptide chains form a first functional target binding site and the VD2 domains on the first and second polypeptide chains form a second functional target binding site.

14. The bispecific binding protein of claim 13, wherein the bispecific binding protein comprises two first polypeptide chains and two second polypeptide chains that form four functional target binding sites.

15. The bispecific binding protein of claim 13, wherein a variable domain that forms a functional target binding site for target one comprises CDRs 1-3 from the amino acid sequence of SEQ ID NO: 32, paired with CDRs 1-3 from the amino acid sequence of SEQ ID NO: 33, and a variable domain that forms a functional target binding site for target two comprises CDRs 1-3 from the amino acid sequence of SEQ ID NO: 34, paired with CDRs 1-3 from the amino acid sequence of SEQ ID NO: 35.

16. The bispecific binding protein of claim 13, wherein a variable domain that forms a functional target binding site for target one comprises the amino acid sequence of SEQ ID NO: 32, paired with the amino acid sequence of SEQ ID NO: 33, and a variable domain that forms a functional target binding site for target two comprises the amino acid sequence of SEQ ID NO: 34, paired with the amino acid sequence of SEQ ID NO: 35.

17. The bispecific binding protein of claim 15, wherein the bispecific binding protein comprises an amino acid sequence selected from the group consisting of SEQ ID NOs: 40-83.

18. The bispecific binding protein of claim 13, wherein X1 comprises at least one of the amino acid sequences of SEQ ID NOs: 21, 22, 13, 14, 29, and 30.

19. The bispecific binding protein of claim 1, wherein the bispecific binding protein is acid sensitive such that it is cleaved in an acidic environment.

20. A bispecific binding protein conjugate comprising the bispecific binding protein of claim 1, linked to an agent selected from the group consisting of an immunoadhesion molecule, an imaging agent, a therapeutic agent, and a cytotoxic agent.

21. The bispecific binding protein conjugate of claim 20, wherein the bispecific binding protein conjugate is acid sensitive such that the agent is released in an acidic environment.

22. A pharmaceutical composition comprising the bispecific binding protein of claim 1, and a pharmaceutically acceptable carrier.

23. A pharmaceutical composition comprising the bispecific binding protein conjugate of claim 20, and a pharmaceutically acceptable carrier.

24. The pharmaceutical composition of claim 22, further comprising at least one additional therapeutic agent.

25. The pharmaceutical composition of claim 24, wherein the at least one additional therapeutic agent is an inhibitor of B cell activation and/or an inhibitor of B cell proliferation and/or an inducer of B cell death.

26. The pharmaceutical composition of claim 25, wherein the inhibitor is an inhibitor of B lymphocyte stimulator (BLys).

27. The pharmaceutical composition of claim 26, wherein the inhibitor is selected from the group consisting of belimumab, tabalumab, atacicept and blisibimod.

28. An isolated nucleic acid encoding the bispecific binding protein of claim 1.

29. A vector comprising the isolated nucleic acid of claim 28.

30. A host cell comprising the vector of claim 25.

31. A method of producing a bispecific binding protein, the method comprising the step of culturing the host cell of claim 30 in culture medium under conditions sufficient to produce the bispecific binding protein.

32. A method of determining a patient's reactivity to a therapeutic agent that is capable of modulating the activity of a TLR, the method comprising the steps of:
(a) obtaining a cell sample from a patient;
(b) treating a first portion of the cell sample with a therapeutic agent in the presence of the bispecific binding protein of claim 1;
(c) treating a second portion of the cell sample with the therapeutic agent in the absence of the bispecific binding protein of claim 1; and
(d) measuring cell proliferation and/or cell death in the samples of steps (b) and (c);
wherein a difference in cell proliferation and/or cell death in the two cell samples is indicative of the patient's reactivity to the therapeutic agent.

33. The method of claim 32, wherein the method is used to determine the eligibility of the patient for a clinical trial to assess the efficacy of the therapeutic agent.

34. The method of claim 32, wherein the patient is suspected of having an autoimmune disease that comprises activation of a TLR.

35. The method of claim 32, wherein the cell sample comprises a B cell sample.

36. The method of claim 34, wherein the TLR is TLR7 and/or TLR9.

37. The method of claim 36, wherein the autoimmune disease is selected from the group consisting of systemic lupus erythematosus (SLE), lupus nephritis, discoid lupus, neonatal lupus, Sjogren's disease, dermatomyostitis and systemic sclerosis.

38. A method for activating or inhibiting TLR9 responsive cells in a patient in need of TLR9 activation or TLR9 inhibition, respectively, the method comprising the step of administering the pharmaceutical composition of claim 22 to a patient in need thereof.

39. A method for treating a patient in need of TLR9 activation or TLR9 inhibition, the method comprising the steps of (a) obtaining a cell sample comprising TLR9 responsive cells from the patient; (b) treating the patient's TLR9 responsive cells with the pharmaceutical composition of claim 22; and (c) reintroducing the treated cells into the patient.

40. A method of identifying an inhibitor or stimulator of TLR signaling, the method comprising the steps of
a) combining a test agent, a B cell, and the bispecific binding protein of claim 1 under conditions suitable for detecting a bispecific binding protein-induced response in the B cell; and
b) determining the ability of the test agent to inhibit or stimulate the bispecific binding protein-induced response in the B cell,
wherein an inhibition of the bispecific binding protein-induced response is indicative that the test agent is an inhibitor of TLR signaling, or
wherein an stimulation of the bispecific binding protein-induced response is indicative that the test agent is a stimulator of TLR signaling.

41. The method of claim 40, wherein the TLR is selected from the group consisting of TLR7 and TLR9.

42. The method of claim 40, wherein the bispecific binding protein-induced response comprises a response selected from the group consisting of cell proliferation and/or cell death.
